SEQUENCE LISTING (39 sequences)

SEQ ID NO: 1 - DNA, 1053 bp, synthetic construct
  misc_feature 1..1053: note = LIG3-4 meganuclease
  source 1..1053: mol_type = other DNA
atgaacaccaagtacaacaaggagttcctgctctacctggccggcttcgtggacggcgacggctccatcaaggcgcagatcaagccgaaccagtcctgcaagttcaagcaccagctctccctgaccttccaggtgacccagaagacgcagaggcgctggttcctcgacaagctggtcgacgagatcggggtgggctacgtctacgaccgcgggtcggtgtccgactacgagctctcccagatcaagcccctgcacaacttcctcacccagctccagccgttcctcaagctgaagcagaagcaggcgaacctcgtcctgaagatcatcgagcagctcccctcggccaaggagtccccggacaagttcctggaggtgtgcacgtgggtcgaccagatcgcggccctcaacgacagcaagacccgcaagacgacctcggagacggtgcgggcggtcctggactccctcccaggatccgtgggaggtctatcgccatctcaggcatccagcgccgcatcctcggcttcctcaagcccgggttcagggatctccgaagcactcagagctggagcaactaagtccaaggaattcctgctctacctggccggcttcgtggacggcgacggctccatcatcgcgtccatcaagccgcgccagtgctacaagttcaagcacgagctccgcctggagttcaccgtgacccagaagacgcagaggcgctggttcctcgacaagctggtcgacgagatcggggtgggctacgtctacgaccgcgggtcggtgtccgactaccgcctctcccagatcaagcccctgcacaacttcctcacccagctccagccgttcctcaagctgaagcagaagcaggcgaacctcgtcctgaagatcatcgagcagctcccctcggccaaggagtccccggacaagttcctggaggtgtgcacgtgggtcgaccagatcgcggccctcaacgacagcaagacccgcaagacgacctcggagacggtgcgggcggtcctggactccctcagcgagaagaagaagtcgtccccctga

SEQ ID NO: 2 - protein, 350 aa, synthetic construct
  REGION 1..350: note = LIG3-4 meganuclease
MNTKYNKEFLLYLAGFVDGDGSIKAQIKPNQSCKFKHQLSLTFQVTQKTQRRWFLDKLVDEIGVGYVYDRGSVSDYELSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGATKSKEFLLYLAGFVDGDGSIIASIKPRQCYKFKHELRLEFTVTQKTQRRWFLDKLVDEIGVGYVYDRGSVSDYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 3 - DNA, 1053 bp, synthetic construct
  misc_feature 1..1053: note = MHP14+ meganuclease
  source 1..1053: mol_type = other DNA
atgaacaccaagtacaacaaggagttcctgctctacctggccggcttcgtggacggcgacggctccatcatcgcgcagatcaagccgaaccagtcctacaagttcaagcaccagctcatgctgaccttcaccgtgacccagaagacgcagaggcgctggttcctcgacaagctggtcgacgagatcggggtgggcaaggtccgcgaccgcgggtcggtgtccgactacatcctctcccagatcaagcccctgcacaacttcctcacccagctccagccgttcctcaagctgaagcagaagcaggcgaacctcgtcctgaagatcatcgagcagctcccctcggccaaggagtccccggacaagttcctggaggtgtgcacgtgggtcgaccagatcgcggccctcaacgacagcaagacccgcaagacgacctcggagacggtgcgggcggtcctggactccctcccaggatccgtgggaggtctatcgccatctcaggcatccagcgccgcatcctcggcttcctcaagcccgggttcagggatctccgaagcactcagagctggagcaactaagtccaaggaattcctgctctacctggccggcttcgtggacggcgacggctccatcatcgcggcgatcaagccgaaccagtcctacaagttcaagcaccagctctccctgaccttcaccgtgacccagaagacgcagaggcgctggttcctcgacaagctggtcgacgagatcggggtgggctacgtccgcgaccaggggtcggtgtcccactaccagctctcccagatcaagcccctgcacaacttcctcacccagctccagccgttcctcaagctgaagcagaagcaggcgaacctcgtcctgaagatcatcgagcagctcccctcggccaaggagtccccggacaagttcctggaggtgtgcacgtgggtcgaccagatcgcggccctcaacgacagcaagacccgcaagacgacctcggagacggtgcgggcggttctagactccctcagcgagaagaagaagtcgtccccctga

SEQ ID NO: 4 - protein, 350 aa, synthetic construct
  REGION 1..350: note = MHP14+ meganuclease
MNTKYNKEFLLYLAGFVDGDGSIIAQIKPNQSYKFKHQLMLTFTVTQKTQRRWFLDKLVDEIGVGKVRDRGSVSDYILSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGATKSKEFLLYLAGFVDGDGSIIAAIKPNQSYKFKHQLSLTFTVTQKTQRRWFLDKLVDEIGVGYVRDQGSVSHYQLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 5 - DNA, 41 bp, synthetic construct
  misc_feature 1..41: note = dephosphorylated adapter
  misc_difference 38..41: note = n is a, c, g, or t
  source 1..41: mol_type = other DNA
ggttgacatgctggattgagacttccctgcaggacgtnnnn

SEQ ID NO: 6 - DNA, 24 bp, synthetic construct
  misc_feature 1..24: note = primer A
  source 1..24: mol_type = other DNA
gttgacatgctggattgagacttc

SEQ ID NO: 7 - DNA, 21 bp, synthetic construct
  misc_feature 1..21: note = primer B
  source 1..21: mol_type = other DNA
caagcagaagacggcatacga

SEQ ID NO: 8 - DNA, 61 bp, synthetic construct
  misc_feature 1..61: note = Illumina-compatibe adapter
  source 1..61: mol_type = other DNA
aatgatacggcgaccaccgagatctacactctttccctacacgacgctcttccgatctgca

SEQ ID NO: 9-10 - skipped (2 entries)

SEQ ID NO: 11 - DNA, 22 bp, synthetic construct
  misc_feature 1..22: note = Figure 2 5'-3' SEQUENCE
  source 1..22: mol_type = other DNA
caagaagaagcacgtcagctta

SEQ ID NO: 12 - DNA, 22 bp, synthetic construct
  misc_feature 1..22: note = Figure 2 3'-5' SEQUENCE
  source 1..22: mol_type = other DNA
gttcttcttcgtgcagtcgaat

SEQ ID NO: 13 - DNA, 22 bp, synthetic construct
  misc_feature 1..22: note = LIG3-4 Intended Recognition Sequence
  source 1..22: mol_type = other DNA
atatacctcacacgtacgcgta

SEQ ID NO: 14 - DNA, 22 bp, synthetic construct
  misc_feature 1..22: note = MHP14+ Intended Recognition Sequence
  source 1..22: mol_type = other DNA
caaacagattcacgtcagattt

SEQ ID NO: 15 - DNA, 22 bp, synthetic construct
  misc_feature 1..22: note = LIG3-4 variant recognition sequence, -11 C
  source 1..22: mol_type = other DNA
ctatacctcacacgtacgcgta

SEQ ID NO: 16 - DNA, 22 bp, synthetic construct
  misc_feature 1..22: note = LIG3-4 variant recognition sequence, -7C
  source 1..22: mol_type = other DNA
atatccctcacacgtacgcgta

SEQ ID NO: 17 - DNA, 22 bp, synthetic construct
  misc_feature 1..22: note = LIG3-4 variant recognition sequence, -2G
  source 1..22: mol_type = other DNA
atatacctcgcacgtacgcgta

SEQ ID NO: 18 - DNA, 22 bp, synthetic construct
  misc_feature 1..22: note = LIG3-4 variant recognition sequence, -1T
  source 1..22: mol_type = other DNA
atatacctcatacgtacgcgta

SEQ ID NO: 19 - DNA, 22 bp, synthetic construct
  misc_feature 1..22: note = LIG3-4 variant recognition sequence, +8T
  source 1..22: mol_type = other DNA
atatacctcacacgtacgtgta

SEQ ID NO: 20 - DNA, 22 bp, synthetic construct
  misc_feature 1..22: note = LIG3-4 variant recognition sequence,-7C, +8T
  source 1..22: mol_type = other DNA
atatccctcacacgtacgtgta

SEQ ID NO: 21 - DNA, 22 bp, synthetic construct
  misc_feature 1..22: note = LIG3-4 variant recognition sequence, -11C, -7C, -2G, -1T, +8T
  source 1..22: mol_type = other DNA
ctatccctcgtacgtacgtgta

SEQ ID NO: 22 - DNA, 22 bp, synthetic construct
  misc_feature 1..22: note = LIG3-4 variant recognition sequence, -11C, -7C, -1T, +8T
  source 1..22: mol_type = other DNA
ctatccctcatacgtacgtgta

SEQ ID NO: 23 - DNA, 22 bp, synthetic construct
  misc_feature 1..22: note = MHP14+ variant recognition sequence, -3A
  source 1..22: mol_type = other DNA
caaacagaatcacgtcagattt

SEQ ID NO: 24 - DNA, 22 bp, synthetic construct
  misc_feature 1..22: note = MHP14+ variant recognition sequence, -2G
  source 1..22: mol_type = other DNA
caaacagatgcacgtcagattt

SEQ ID NO: 25 - DNA, 22 bp, synthetic construct
  misc_feature 1..22: note = MHP14+ variant recognition sequence, -1T
  source 1..22: mol_type = other DNA
caaacagatttacgtcagattt

SEQ ID NO: 26 - DNA, 22 bp, synthetic construct
  misc_feature 1..22: note = MHP14+ variant recognition sequence, +2A
  source 1..22: mol_type = other DNA
caaacagattcaagtcagattt

SEQ ID NO: 27 - DNA, 22 bp, synthetic construct
  misc_feature 1..22: note = MHP14+ variant recognition sequence, +7T
  source 1..22: mol_type = other DNA
caaacagattcacgtcatattt

SEQ ID NO: 28 - DNA, 22 bp, synthetic construct
  misc_feature 1..22: note = MHP14+ variant recognition sequence, +8G
  source 1..22: mol_type = other DNA
caaacagattcacgtcaggttt

SEQ ID NO: 29 - DNA, 22 bp, synthetic construct
  misc_feature 1..22: note = MHP14+ variant recognition sequence, +11G
  source 1..22: mol_type = other DNA
caaacagattcacgtcagattg

SEQ ID NO: 30 - DNA, 22 bp, synthetic construct
  misc_feature 1..22: note = MHP14+ variant recognition sequence, +11A
  source 1..22: mol_type = other DNA
caaacagattcacgtcagatta

SEQ ID NO: 31 - DNA, 22 bp, synthetic construct
  misc_feature 1..22: note = MHP14+ variant recognition sequence, -3A, -2G, -1T, +2A, +7T, +8G, +11G
  source 1..22: mol_type = other DNA
caaacagaagtaagtcatgttg

SEQ ID NO: 32 - DNA, 22 bp, synthetic construct
  misc_feature 1..22: note = MHP14+ variant recognition sequence, -3A, -2G, -1T, +2A, +7T, +8G, +11A
  source 1..22: mol_type = other DNA
caaacagaagtaagtcatgtta

SEQ ID NO: 33 - DNA, 22 bp, synthetic construct
  misc_feature 1..22: note = MHP14+ variant recognition sequence, 3A, -2G, -1T, +7T, +8G, +11G
  source 1..22: mol_type = other DNA
caaacagaagtacgtcatgttg

SEQ ID NO: 34 - DNA, 22 bp, synthetic construct
  misc_feature 1..22: note = MHP14+ variant recognition sequence, -2G, -1T, +2A, +7T, +8G, +11G
  source 1..22: mol_type = other DNA
caaacagatgtaagtcatgttg

SEQ ID NO: 35 - DNA, 22 bp, synthetic construct
  misc_feature 1..22: note = MHP14+ variant recognition sequence,-2G, -1T, +7T, +8G, +11G
  source 1..22: mol_type = other DNA
caaacagatgtacgtcatgttg

SEQ ID NO: 36 - DNA, 3716 bp, Saccharomyces cerevisiae
  source 1..3716: mol_type = genomic DNA
ctactagaaagaagggcgtcaaaaatctgctatgctattacgaaattactctataggttagaatgtcatcccataggtatggcccttttaaagaagtatctccacaatcaattgcgagaagcctatctagaatcaaaacgacactttatttccaaaaagggagattcgaccaacacttcctctaccattgcatcatcatctttcgctggcgcatctgttcctctatcttcaaacgaatcaggaatgctaaacggcttgaagcaaattaacgaacaacaagaatctacattagaaaccactcaaaaggaagactagtaacgccgtatcgtgattaacgtattacataagttacaggattcatgcttatgggttagctatttcgcccaatgtgtccatctgacattactattttgcattttaatttaattagaacttgactagcgcactaccagtatatcatctcatttccgtaaataccaaatgtattatatattgaaagcttttgaccaggttattataaaagaaacttcatgctcgaaaaagatcatttcgaaaagttgcctagtttcatgaaattttaaagcagtttatataaattttaccttttgatgcggaattgactttttcttgaataatacataacttttcttaaaagaatcaaagacagataaaatttaagagatattaaatattagtgagaagccgagaattttgtaacaccaacataacactgacatctttaacaacttttaattatgatacatttcttacgtcatgattgattattacagctatgctgacaaatgactcttgttgcatggctacgaaccgggtaatactaagtgattgactcttgctgaccttttattaagaactaaatggacaatattatggagcatttcatgtataaattggtgcgtaaaatcgttggatctctcttctaagtacatcctactataacaatcaagaaaaacaagaaaatcggacaaaacaatcaagtatggattctagaacagttggtatattaggagggggacaattgggacgtatgattgttgaggcagcaaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcagattgatacaagacaaatatattcaaaaagagcatttaatcaaaaatggtatagcagttacccaaagtgttcctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatgggcaccatttactaaagaattagcagtcatgattgtgagatctgttaacggtttagtgttttcttacccaattgtagagactatccacaaggacaatatttgtgacttatgttatgcgcctgctagagttccggactccgttcaacttaaggcgaagttgttggcagaaaatgcaatcaaatcttttcccggttgtggtatatttggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacaggtagaactgatattccaatcaaaatctctgtcgctcaaaagttggacttggaagcaatggtcaaaccattggttggaatcatcatgggatcagactctgacttgccggtaatgtctgccgcatgtgcggttttaaaagattttggcgttccatttgaagtgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaattgtgcaaatgcctagaggtgttccagtagctaccgtcgctattaataatagtacgaacgctgcgctgttggctgtcagactgcttggcgcttatgattcaagttatacaacgaaaatggaacagtttttattaaagcaagaagaagaagttcttgtcaaagcacaaaagttagaaactgtcggttacgaagcttatctagaaaacaagtaatatataagtttattgatatacttgtacagcaaataattataaaatgatatacctattttttaggctttgttatgattacatcaaatgtggacttcatacatagaaatcaacgcttacaggtgtccttttttaagaatttcatacataagatcacttattatacatacatacatatccagtaacaagaagcaaggaataattacctgcttaagtctgcgattaaaaaaataacgtttcgatacagttcatataaggcggctcaatgcagaaccgaggatagcgctacgtcaggatatctttgtagttcccaaatataaatgcgacaatatagtttctttctttcatatcaataatatccttttctccactgaaatcacgaatcaaacctggagcaaaaactaaagccaaattataaagcgtcattcgattccagtgactgtaccgtgtaaccttttctatatgttcactcagtactcttaacaccctataatgttcccttggaaggtcttccaatatgttttttaaagcgctcttgctcgacatataagtgtccgaattctttgcttctaaggacaactttcctccaacaaatggcaagttttccatcatttttttagatttaactaacctcatcaacggctcgtatatttgaaaggtaaagatagggttggggagctttcttaagtatcgcttcaacacaccagtaacaacgttgagatcttgttccgttaaaatatttggcgtttcggtattttgttgtactttccatgcagaaaattgcttttctatttcttctatgactagctgggaacctgattttctataaatgccctccgatctcatattttcttcgtctgattcaataaaatctatgcagacagatagtatcatcggtatttcattgttttcataattgcacctagcaacgagacttgaaccatacaaattgcttccatccaaatattcttctccatctttactttgtcccatatttgcatcg

SEQ ID NO: 37 - DNA, 22 bp, synthetic construct
  misc_feature 1..22: note = MS26 intended recognition sequence
  source 1..22: mol_type = other DNA
gatggtgacgtacgtgccctac

SEQ ID NO: 38 - DNA, 16824 bp, synthetic construct
  misc_feature 1..16824: note = plasmid PHP57712
  misc_difference 12027: note = n is a, c, g, or t
  source 1..16824: mol_type = other DNA
ggagatccaagcttggcgcgccggcctctgcctgcgttctgctgtggaagttcctattccgaagttcctattctccagaaagtataggaacttcacatgctgcctcgtgcaagtcacgatctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaacagaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcagccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatattcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccatcaggggcagatctaggcgcgcgccatatacctcacacgtacgcgtagatggtgacgtacgtgccctaccaaacagattcacgtcagatttgaagttcctattccgaagttcctattctacatagagtataggaacttccgatatcactgcagtggccggcggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatcaccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaacggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtactttctagacgtacgtctttccacaatacataactattaattaatcttaaataaataaaggataaaatatttttttttcttcataaagttaaaatatgttattttttgtttagatgtatattcgaataaatctaaatatatgataatgattttttatattgattaaacatataatcaatattaaatatgatatttttttatataggttgtacacataattttataaggataaaaaatatgataaaaataaattttaaatatttttatatttacgagaaaaaaaaatattttagccataaataaatgaccagcatattttacaaccttagtaattcataaattcctatatgtatatttgaaattaaaaacagataatcgttaagggaaggaatcctacgtcatctcttgccatttgtttttcatgcaaacagaaagggacgaaaaaccacctcaccatgaatcactcttcacaccatttttactagcaaacaagtctcaacaactgaagccagctctctttccgtttctttttacaacactttctttgaaatagtagtattttttttcacatgatttattaacgtgccaaaagatgcttattgaatagagtgcacatttgtaatgtactactaattagaacatgaaaaagcattgttctaacacgataatcctgtgaaggcgttaactccaaagatccaatttcactatataaattgtgacgaaagcaaaatgaattcacatagctgagagagaaaggaaaggttaactaagaagcaatacttcagcggccgcttctagctagctagggtttgggtagtgagtgtaataaagttgcaaagtttttggttaggttacgttttgaccttattattatagttcaaagggaaacattaattaaaggggattatgaagtgggctctcttgattcttggatgaggatcttactgggtgaattgagctgcttagctatggatcccacagttctacccatcaataagtgcttttgtggtagtcttgtggcttccatatctggggagcttcatttgcctttatagtattaaccttctccaagaacaaagagagcccacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatgagccgtaaaggttcaatacaacgagtgcttgttttcttagggacaagcattgtacttatgtatgattctgtgtaaccatgagtcttccacgttgtactaatgtgaagggcaaaaataaaacacagaacaagttcgtttttctcaaataatgtgaaggtagaaaatggaaccatgcctcctctcttgcatgtgatttaaaatattagcagatggtacgtcgagtcgacctgcaggtcgactcgacgtacgtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggcgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgactagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccggtctaggccaaggccgcgaagttaaaagcaatgttgtcacttgtacgtactaacacatgatgtgatagtttatgctagctagctataacataagctgtctctgagtgtgttgtatattaataaagatcatcactggtgaatggtgatcgtgtacgtaccctacttagtaggcaatggaagcacttagagtgtgctttgtgcatggccttgcctctgttttgagacttttgtaatgttttcgagtttaaatctttgcctttgcgtacgtgggcggatcccctgca

SEQ ID NO: 39 - DNA, 16822 bp, synthetic construct
  misc_feature 1..16822: note = plasmid PHP62552
  misc_difference 9373: note = n is a, c, g, or t
  source 1..16822: mol_type = other DNA
gaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcagccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagtgggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatattcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccatcaggggcagatctaggcgcgccgatggtgacgtacgtgccctaccaaacagatgtacgtcatgttgctatccctcatacgtacgtgtagaagttcctattccgaagttcctattctacatagagtataggaacttccgatatcactgcagtggccggcggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatcaccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaacggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtactttctagacgtacgtctttccacaatacataactattaattaatcttaaataaataaaggataaaatatttttttttcttcataaagttaaaatatgttattttttgtttagatgtatattcgaataaatctaaatatatgataatgattttttatattgattaaacatataatcaatattaaatatgatatttttttatataggttgtacacataattttataaggataaaaaatatgataaaaataaattttaaatatttttatatttacgagaaaaaaaaatattttagccataaataaatgaccagcatattttacaaccttagtaattcataaattcctatatgtatatttgaaattaaaaacagataatcgttaagggaaggaatcctacgtcatctcttgccatttgtttttcatgcaaacagaaagggacgaaaaaccacctcaccatgaatcactcttcacaccatttttactagcaaacaagtctcaacaactgaagccagctctctttccgtttctttttacaacactttctttgaaatagtagtattttttttcacatgatttattaacgtgccaaaagatgcttattgaatagagtgcacatttgtaatgtactactaattagaacatgaaaaagcattgttctaacacgataatcctgtgaaggcgttaactccaaagatccaatttcactatataaattgtgacgaaagcaaaatgaattcacatagctgagagagaaaggaaaggttaactaagaagcaatacttcagcggccgcttctagctagctagggtttgggtagtgagtgtaataaagttgcaaagtttttggttaggttacgttttgaccttattattatagttcaaagggaaacattaattaaaggggattatgaagtgggctctcttgattcttggatgaggatcttactgggtgaattgagctgcttagctatggatcccacagttctacccatcaataagtgcttttgtggtagtcttgtggcttccatatctggggagcttcatttgcctttatagtattaaccttctccaagaacaaagagagcccacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatgagccgtaaaggttcaatacaacgagtgcttgttttcttagggacaagcattgtacttatgtatgattctgtgtaaccatgagtcttccacgttgtactaatgtgaagggcaaaaataaaacacagaacaagttcgtttttctcaaataatgtgaaggtagaaaatggaaccatgcctcctctcttgcatgtgatttaaaatattagcagatggtacgtcgagtcgacctgcaggtcgactcgacgtacgtcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgctagtcgactaagtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatgtacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaagtgactcgaggtcatcaattactccaagctacgtatttgggagttcgtggagtacaagaagacgatatacctagacggtgacatccaagtatttggaaacatagaccacttgtttgatctgcctgataattatttctatgcggtgatggattgtttctgcgagaagacttggagccacacccctcagttccagattgggtactgccaacagtgccctgataaggttcaatggccctctcactttggttccaaacctcctctatatttcaatgctggcatgtttgtttatgagcctaatctcgacacctaccgtgatcttctccaaactgtccaactcaccaagcccacttcttttgctgagcaggactttctcaacatgtacttcaaggacaagtacaagccaataccgaacatgtacaaccttgtgctggccatgttgtggcgtcaccctgaaaatgttgaacttgataaagttcaagtggttcattactgtgctgctgggtctaagccttggaggttcactgggaagtaactgcaggtcatcaactactccaagctccgtatatgggagtttgtggagtacagcaagatgatatacttggacggagacattgaggtatatgagaacatagaccacctatttgacctacctgatggtaacttttacgctgtgatggattgtttctgcgagaagacatggagtcacacccctcagtacaaggtgggttactgccagcaatgcccggagaaggtgcggtggcccaccgaattgggtcagcccccttctctttacttcaacgctggcatgttcgtgttcgaacccaacatcgccacctatcatgacctattgaaaacggtgcaagtcaccactcccacctcgttcgctgaacaagatttcttgaacatgtacttcaaggacatttacaagccaatccctttaaattacaatcttgtcctcgccatgctgtggcgccacccggaaaacgttaaattagaccaagtcaaggttgttcactattgcgcagcggggtccaagccatggagatatacggggaagtagcctaggcgtacgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtcctaggctacttccccgtatatctccatggcttggaccccgctgcgcaatagtgaacaaccttgacttggtctaatttaacgttttccgggtggcgccacagcatggcgaggacaagattgtaatttaaagggattggcttgtaaatgtccttgaagtacatgttcaagaaatcttgttcagcgaacgaggtgggagtggtgacttgcaccgttttcaataggtcatgataggtggcgatgttgggttcgaacacgaacatgccagcgttgaagtaaagagaagggggctgacccaattcggtgggccaccgcaccttctccgggcattgctggcagtaacccaccttgtactgaggggtgtgactccatgtcttctcgcagaaacaatccatcacagcgtaaaagttaccatcaggtaggtcaaataggtggtctatgttctcatatacctcaatgtctccgtccaagtatatcatcttgctgtactccacaaactcccatatacggagcttggagtagttgatgacctgcagttacttcccagtgaacctccaaggcttagacccagcagcacagtaatgaaccacttgaactttatcaagttcaacattttcagggtgacgccacaacatggccagcacaaggttgtacatgttcggtattggcttgtacttgtccttgaagtacatgttgagaaagtcctgctcagcaaaagaagtgggcttggtgagttggacagtttggagaagatcacggtaggtgtcgagattaggctcataaacaaacatgccagcattgaaatatagaggaggtttggaaccaaagtgagagggccattgaaccttatcagggcactgttggcagtacccaatctggaactgaggggtgtggctccaagtcttctcgcagaaacaatccatcaccgcatagaaataattatcaggcagatcaaacaagtggtctatgtttccaaatacttggatgtcaccgtctaggtatatcgtcttcttgtactccacgaactcccaaatacgtagcttggagtaattgatgacctcgagtcacttcccagtgtacctccaaggcttagacccagcagcacagtagtgaaccactttaactttgtcaagctcaacgttctcagggtgacgccacagcatggccagcacaagattgtagacattaggaattggcctatatttgtccttgaagtacatgttcaaaaaatcctgttcagcaaaggaagtgggctgggtgacttggactgtttgaaggaggtcacggtaagtagccaaattgggctcatacacaaacatgccagcattgaaatagagaggaggtttgggcccaaagtgagtgggccactgaaccttatgggggcactgctggcagtaaccgatctgatattgtttagtgtggccccaagttggctcacagaaacagtccatcaccgcatagaagtagttatcaggcaagtcaaacaagtggtcaatgttgtcaaaaacttggatatcaccgtctaggtatatcatcttgctgtactccacaaactcccaaatacgtagcttggaatagttgatgacttagtcgactagcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccggtctaggccaaggccgcgaagttaaaagcaatgttgtcacttgtacgtactaacacatgatgtgatagtttatgctagctagctataacataagctgtctctgagtgtgttgtatattaataaagatcatcactggtgaatggtgatcgtgtacgtaccctacttagtaggcaatggaagcacttagagtgtgctttgtgcatggccttgcctctgttttgagacttttgtaatgttttcgagtttaaatctttgcctttgcgtacgtgggcggatcccctgcaggagatccaagcttggcgcgccggcctctgcctgcgttctgctgtggaagttcctattccgaagttcctattctccagaaagtataggaacttcacatgctgcctcgtgcaagtcacgatctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaaca

(19) United States
(12) Patent Application Publication
(10) Pub. No.: US 2024/0191247 A1
(43) Pub. Date: Jun. 13, 2024

(54) METHODS FOR THE IDENTIFICATION OF VARIANT RECOGNITION SITES FOR RARE-CUTTING ENGINEERED DOUBLE-STRAND-BREAK-INDUCING AGENTS AND COMPOSITIONS AND USES THEREOF

(71) Applicants: CORTEVA AGRISCIENCE LLC, INDIANAPOLIS, IN (US); PIONEER HI-BRED INTERNATIONAL, INC., JOHNSTON, IA (US)

(72) Inventors: STEPHANE DESCHAMPS, WEST DES MOINES, IA (US); JAMES ENGLISH, SAN RAMON, CA (US); ZHONGSEN LI, HOCKESSIN, DE (US); JOSHUA K. YOUNG, JOHNSTON, IA (US); VICTOR LLACA, CLIVE, IA (US)

(73) Assignees: CORTEVA AGRISCIENCE LLC, INDIANAPOLIS, IN (US); PIONEER HI-BRED INTERNATIONAL, INC., JOHNSTON, IA (US)

(21) Appl. No.: 18/544,882

(22) Filed: Dec. 19, 2023

Related U.S. Application Data
(62) Division of application No. 16/415,179, filed on May 17, 2019, now abandoned.
Application No. 16/415,179 is a continuation of application No. 14/775,777, filed on Sep. 14, 2015, now Pat. No. 10,329,574.
(60) Provisional application No. 61/777,238, filed on Mar. 12, 2013.

(51) Int. Cl. C12N 15/82; A01H 6/14; A01H 6/46; A01H 6/82; C12N 9/22; C12Q 1/6811

(52) U.S. Cl. CPC C12N 15/8213; A01H 6/14; A01H 6/46; A01H 6/82; C12N 9/22; C12N 15/8241; C12Q 1/6811

(57) ABSTRACT

Methods for the identification of variant recognition sites for rare cutting engineered double strand break inducing agents and compositions thereof are provided. Further provided are nucleic acid constructs, yeast, plants, plant cells, explants, seeds and grain having the of variant recognition sites. Various methods of identifying variant recognition sites with increased substrate activity for a rare cutting engineered double strand break inducing agents are provided.

### CROSS-REFERENCE TO RELATED APPLICATIONS

This application is a divisional of U.S. application Ser. No. 16/415,179, filed May 17, 2019, which is continuation of U.S. application Ser. No. 14/775,777, filed Sep. 14, 2015, now U.S. Pat. No. 10,329,574, issued Jun. 25, 2019, which is a 371 National Stage Entry of PCT/US14/22500 which was filed Mar. 10, 2014, which claims the benefit of U.S. Patent Application Ser. No. 61/777,238, filed Mar. 12, 2013, all of which are herein incorporated by reference in their entireties.

### FIELD OF THE INVENTION

This invention is in the field of molecular biology. More specifically, this invention pertains to methods for identifying and using variant recognition sites for rare-cutting engineered double strand break inducing agents.

### REFERENCE TO SEQUENCE LISTING SUBMITTED ELECTRONICALLY

The official copy of the sequence listing is submitted electronically via EFS-Web as an XML formatted sequence listing with a file named 115465-US-DIV-1_ST26.XML created on Feb. 29, 2024, and having a size of 88 kilobytes and is filed concurrently with the specification. The sequence listing comprised in this XML formatted document is part of the specification and is herein incorporated by reference in its entirety.

### BACKGROUND OF THE INVENTION

Recombinant DNA technology has made it possible to insert foreign DNA sequences into the genome of an organism, thus, altering the organism's phenotype. The most commonly used plant transformation methods are Agrobacterium infection and biolistic particle bombardment in which transgenes integrate into a plant genome in a random fashion and in an unpredictable copy number. Thus, efforts are undertaken to control transgene integration in plants.

Methods for inserting or modifying a DNA sequence into the genome of a variety of organism have been developed and can involve site-specific integration techniques, which rely on homologous recombination (U.S. Pat. No. 7,102,055 issued on Sep. 5, 2006) or designer endonucleases such as meganucleases, zinc finger nucleases or TALENs (US patent publication 2009-0133152 A1, published May 21, 2009).

While these systems have provided useful techniques for targeted insertion of sequences of interest, there remains a need for identifying more recognition sites for rare cutting double strand break inducing agents and for identifying recognition sites with increased activity towards rare cutting double strand beak inducing agents.

### BRIEF SUMMARY OF THE INVENTION

Compositions and methods are provided which employ variant recognition sites for a rare-cutting engineered double-strand-break-inducing agent capable of introducing a rare double strand break in an intended recognition site.

Methods for identifying a variant recognition site for a rare-cutting engineered double-strand-break-inducing agent capable of introducing a rare double strand break in an intended recognition site are provided. One method comprises, a) contacting genomic DNA with a rare-cutting engineered double-strand-break-inducing agent capable of introducing a double-strand break into said genomic DNA, wherein the double-strand break results in a nucleotide overhang, b) ligating a first adapter to said nucleotide overhang, c) shearing the ligated DNA obtained in step (b) and ligating at least one second adapter to the sheared nucleotide end to allow for the amplification and sequencing of genomic DNA fragments surrounding the double strand break, d) aligning nucleotide sequences of the DNA fragments obtained in (c) with a reference genome DNA sequence; and e) identifying a variant recognition site comprising at least one nucleotide base alteration when compared to the intended recognition site of said engineered double-strand break-inducing agent. Another method comprises a method to identify a variant recognition site for a rare-cutting engineered double-strand-break-inducing agent capable of introducing a rare double strand break in an intended recognition site, said method comprising: a) contacting genomic DNA with a rare-cutting engineered double-strand-break-inducing agent capable of introducing a double-strand break into said genomic DNA, wherein the double-strand break results in a blunt end; b) creating a nucleotide overhang from the blunt end of (a); c) ligating a first adapter to the nucleotide overhang of (b); d) shearing the ligated DNA obtained in step (c) and ligating at least one second adapter to the sheared nucleotide end to allow for the amplification and sequencing of genomic DNA fragments surrounding the double strand break; e) aligning nucleotide sequences of the DNA fragments obtained in (d) with a reference genome DNA sequence; and, f) identifying a variant recognition site comprising at least one nucleotide base alteration when compared to the intended recognition site of said engineered double-strand break-inducing agent.

The rare-cutting engineered double-strand-break-inducing agent can be selected from the group consisting of a meganuclease, a zinc finger nuclease, a TAL effector nuclease, a transposase, a Cas endonuclease and a site-specific recombinase. The nucleotide overhang can be a 3′ or 5′ nucleotide overhang.

Further provided are methods to identify a variant recognition site with an improved cleavage activity for a rare-cutting engineered double-strand-break-inducing agent capable of introducing a double strand break in an intended recognition site. The increased activity of the rare-cutting engineered double-strand-break-inducing agent is evidenced by a) a higher percent (%) cleavage of the variant recognition site when compared to the percent (%) cleavage of intended recognition site, wherein the recognition sites are located on genomic DNA; b) a higher percent (%) cleavage of the variant recognition site when compared to the percent (%) cleavage of intended recognition site, wherein the recognition sites are located on plasmid DNA; c) a higher yeast assay score for the variant recognition site when compared to the intended recognition site; or, d) any combination of (a), (b) and (c).

Further provided are methods for targeting the insertion of a polynucleotide of interest to a specific chromosomal site within a plant genome, said method comprising: a) transforming a plant cell or a plant with a DNA fragment comprising a polynucleotide of interest, wherein said genome of said plant cell or plant comprises at least one variant recognition site selected from the group consisting of SEQ ID NOs: 15, 16, 17, 18, 19, 20 and 21 or SEQ ID NOs: 23, 24, 25, 26, 27, 28, 29, 30, 31, 32, 33, 34 and 35; and, b) providing a meganuclease capable of providing a double strand break into the variable recognition site of (a); and, c) selecting said plant cell or plant comprising said polynucleotide of interest integrated into said variant recognition site.

Various compositions include a plant, a seed or a plant cell comprising in its genome a variant recognition site for a rare-cutting engineered double-strand-break-inducing agent capable of introducing a rare double strand break in an intended recognition site.

### BRIEF DESCRIPTION OF THE DRAWINGS AND SEQUENCE LISTING

The invention can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§ 1.821 1.825. The sequence descriptions contain the three letter codes for amino acids as defined in 37 C.F.R. §§ 1.821 1.825, which are incorporated herein by reference.

FIG. 1A Genomic recognition site peak signature from a sample treated with meganuclease. Mapped sequence data originates from and comes back towards the site of cleavage. The directionality of the mapped reads is indicated in the pile-up view and by the arrows. FIG. 1B The mock control contains no enrichment or peak signature as observed for the treated sample.

FIG. 2. The 4 base overlap in the peak recognition site signature corresponds to the overhang generated by the meganuclease and defines the sequence of the genomic variant recognition site. The dashed line defines the overhangs produced by recognition site cleavage.

FIGS. 3A and 3B show the percent DNA base composition of oriented genomic variant recognition sites. Preferred off-nucleotides are outlined while the intended recognition bases are shaded. FIG. 3A Lig3-4 meganuclease DNA base composition of 30 genomic variant recognition sites. The LIG3-4 intended recognition site (SEQ ID NO:13) is shown at the bottom of FIG. 3A. FIG. 3B MHP14+ meganuclease DNA base composition of 254 genomic variant recognition sites. The MHP14+ intended recognition site (SEQ ID NO:14) is shown at the bottom of FIG. 3B.

FIG. 4A Alignment of the intended recognition site for the LIG3-4 meganuclease and LIG3-4 variant recognition sites. Preferred off-nucleotides (outlined) were introduced into the LIG3-4 intended recognition site individually (resulting in the creation of variant recognition sites −11C, −7C, −2G, −1T, +8T corresponding to SEQ ID NOs: 15-19) and in combination (resulting in the creation of variant recognition sites corresponding to SEQ ID NOs:20-22). FIG. 4B Alignment of the intended recognition site for the MHP14+ meganuclease and MHP14+ variant recognition sites. Preferred off-nucleotides (outlined) were introduced into the MHP14+ intended recognition site individually (resulting in the creation of variant recognition sites −3A, −2G, −1T, +2A, +7T, +8G, +11G, +11A corresponding to SEQ ID NOs: 23-30) and in combination (resulting in the creation of variant recognition sites corresponding to SEQ ID NOs: 31-35).

FIGS. 5A and 5B show the comparison of plasmid DNA cleavage activity between the intended recognition site and preferred off-nucleotides individually placed into the intended recognition site for the FIG. 5A Lig3-4 meganuclease and FIG. 5B MHP14+ meganuclease. The percent cleavage activity of the intended recognition site is marked with a dashed line. As a control, at least two bases not preferred in the percent DNA base composition of genomic variant recognition sites were also assayed.

FIGS. 6A and 6B show a comparison of plasmid DNA cleavage activity between the intended recognition site and preferred off-nucleotides placed in combination into the intended recognition site for the FIG. 6A Lig3-4 meganuclease and FIG. 6B MHP14+ meganuclease.

FIG. 7 shows a comparison of real-time PCR amplification plots from genomic variant meganuclease recognition site libraries created with either phosphorylated or non-phosphorylated biotinylated adapters containing a fully degenerate 4 nucleotide 3′ overhang. FIG. 7A shows results for the non-phosphorylated adapter. FIG. 7B shows results for the phosphorylated adapter.

FIG. 8 shows a diagram representing the yeast screening system used to determine the meganuclease activity in yeast. Gene fragments corresponding to the first 1000 nucleotides of the yeast Ade2 coding sequence (Ade2 5′ fragment) and the last 1011 nucleotides of the yeast Ade2 coding sequence (Ade2 3′ fragment) were disrupted by a fragment including the yeast ura3 gene (Ura3) and meganuclease recognition sites for I-SceI.

FIG. 9 shows the numerical scale and corresponding white sectoring of yeast colonies used to quantify meganuclease activity. Since the sectoring phenotype is a qualitative measure of meganuclease activity, a 0-4 numerical scoring system was implemented. A score of 0 indicates that no white sectors (no meganuclease cutting) were observed; a score of 4 indicates completely white colonies (complete cutting of the recognition site); scores of 1-3 indicate intermediate white sectoring phenotypes (and intermediate degrees of recognition site cutting)

FIG. 10A shows a comparison of plasmid DNA cleavage activity between the MHP14+ intended recognition site (SEQ ID NO.: 14), a MHP14+ variant recognition site (SEQ ID NO: 11) that occurs naturally in the maize genome (labeled maize variant recognition site), and MHP14+ variant recognition sites (SEQ ID NOs: 31-35) that are not endogenous to the maize genome. FIG. 10B shows a comparison of the relative copy number of the MHP14+ variant recognition site of SEQ ID NO:11 and the MHP14+ intended recognition site (SEQ ID NO: 14) in mature maize embryos.

FIG. 11A is the map of plasmid PHP57712, and FIG. 11B is the map of plasmid PHP62552.

### SEQUENCES

SEQ ID NO: 1 is the nucleotide sequence encoding the single chain LIG3-4 meganuclease fusion polypeptide.

SEQ ID NO: 2 is the amino acid sequence of the LIG3-4 meganuclease fusion polypeptide.

SEQ ID NO: 3 is the nucleotide sequence encoding the single chain MHP14+ meganuclease.

SEQ ID NO: 4 is the amino acid sequence of the MHP14+ meganuclease.

SEQ ID NO: 5 is the nucleotide sequence of a biotinylated, dephosphorylated adapter designed with a fully-degenerated 4 bp 3′ overhang.

SEQ ID NO: 6 is the nucleotide sequence of recovery primer A.

SEQ ID NO: 7 is the nucleotide sequence of recovery primer B.

SEQ ID NO: 8 is the nucleotide sequence of an Illumina-compatible adapter.

SEQ ID NO: 9 is the nucleotide sequence of a sequence tag.

SEQ ID NO: 10 is the nucleotide sequence of the complement sequence tag of SEQ ID NO: 9.

SEQ ID NO: 11 is the nucleotide sequence of the 5′-3′ sequence shown in FIG. 2.

SEQ ID NO: 12 is the nucleotide sequence of the 3′-5′ sequence shown in FIG. 2.

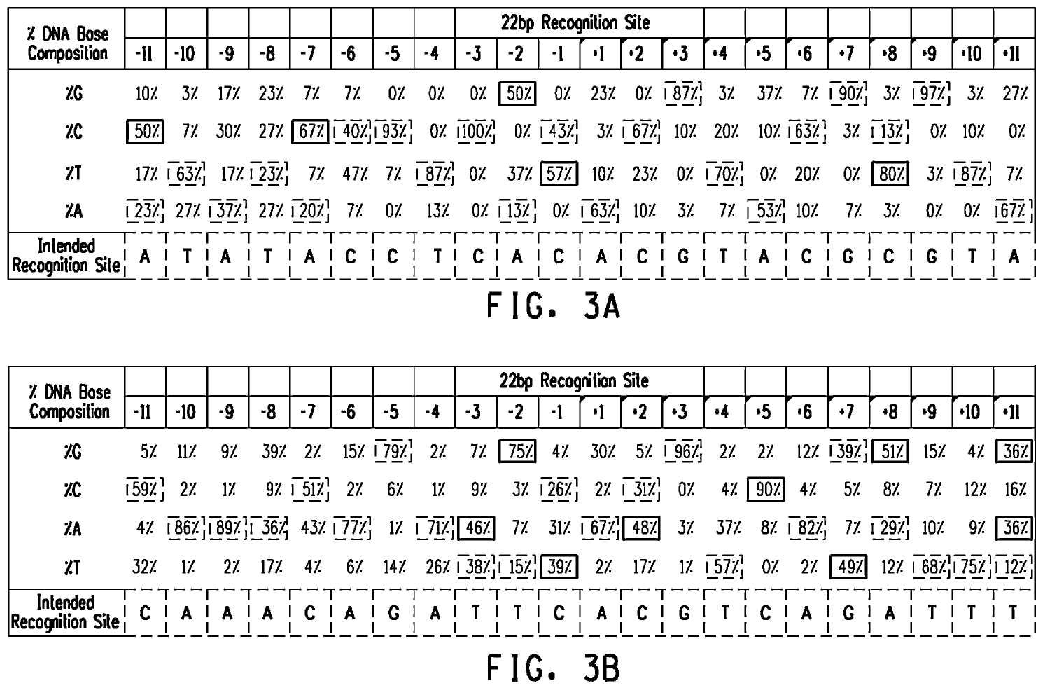

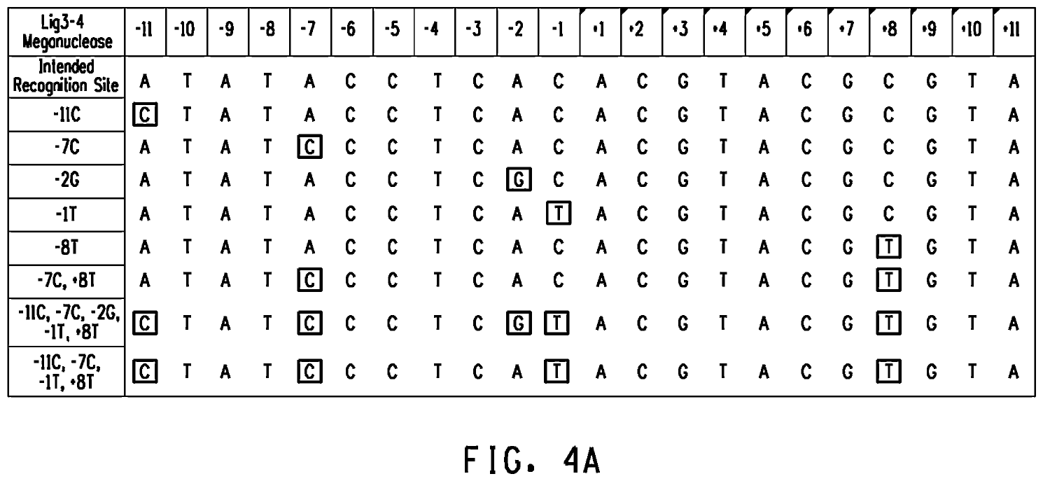

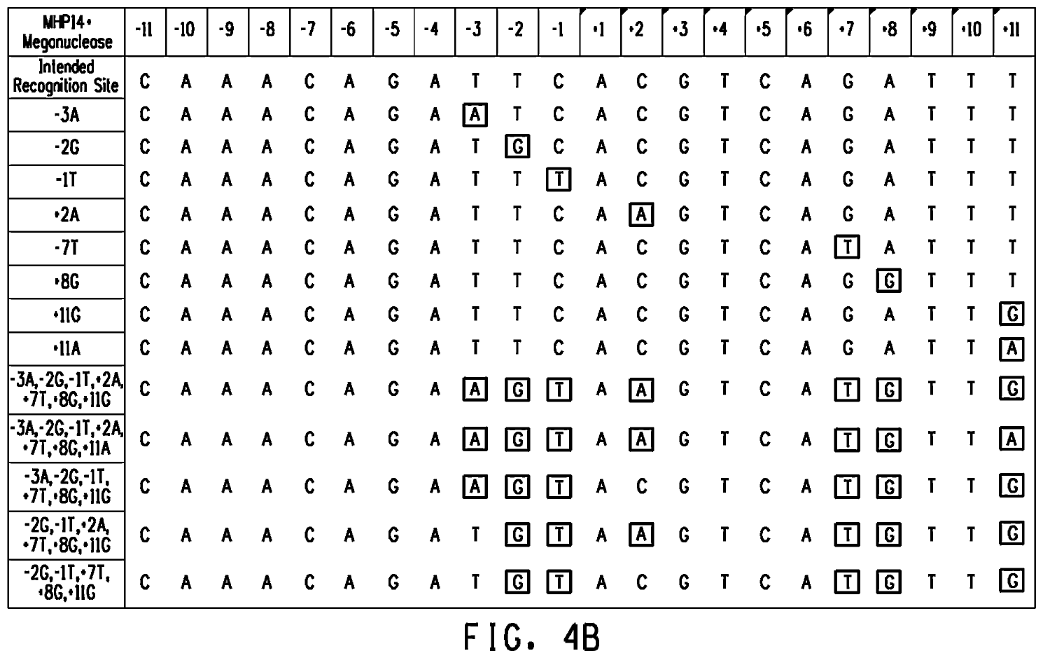

SEQ ID NO: 13 is the nucleotide sequence of the intended recognition site for the LIG3-4 meganuclease (also shown in FIG. 3 A and FIG. 4).

SEQ ID NO: 14 is the nucleotide sequence of the intended recognition site for the MHP14+ meganuclease (also shown in FIG. 3 B and FIG. 4).

SEQ ID NOs:15-22 are nucleotide sequences of variant recognition sites for the LIG3-4 meganuclease.

SEQ ID NOs:23-36 are nucleotide sequences of variant recognition sites for the MHP14+ meganuclease.

SEQ ID NO: 36 is the nucleotide sequence of the Ade2 yeast gene.

SEQ ID NO: 37 is the nucleotide sequence of the intended recognition site for the MS26 meganuclease

SEQ ID NO: 38 is the nucleotide sequence of plasmid PHP57712

SEQ ID NO: 39 is the nucleotide sequence of plasmid PHP62552

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, specific examples of appropriate materials and methods are described herein.

In the context of this disclosure, a number of terms and abbreviations are used. The following definitions are provided.

As used herein, the terms “target site”, “target sequence”, “genomic target site” and “genomic target sequence” are used interchangeably herein and refer to a polynucleotide sequence in the genome of a plant cell or yeast cell that comprises a recognition site for a double-strand-break-inducing agent.

An “artificial target site” is a target sequence that has been introduced into the genome of an organism such as a plant or yeast. Such an artificial target sequence can be identical in sequence to an endogenous or native target sequence in the genome of the organism but can be located in a different position (i.e., a non-endogenous or non-native position) in the genome of the organism.

The terms “endogenous target sequence” and “native target sequence” are used interchangeable herein to refer to a target sequence that is endogenous or native to the genome of a host (such as a plant or yeast) and is at the endogenous or native position of that target sequence in the genome of the host (such as a plant or yeast).

The term “double-strand-break-inducing agent” as used herein refers to any nuclease which produces a double-strand break in the target sequence. Producing the double-strand break in a target sequence or other DNA can be referred to herein as “cutting” or “cleaving” the target sequence or other DNA.

The term “rare-cutting double-strand-break-inducing agent” as used herein refers to any nuclease which produces a double-strand break in a target sequence, but cuts at rare occasions (in contrast to restriction enzymes, for example) in the genome of an organism. Rare-cutting double-strand-break-inducing agents include but are not limited to endonucleases such as meganucleases, (US patent application 2332 and BB1990), zinc finger nucleases (Kim, Y. G., J. Cha, et al. (1996). “Hybrid restriction enzymes: zinc finger fusions to Fok I cleavage;) Cas endonucleases (WO2007/025097 application published Mar. 1, 2007) and TALENs (Christian, M., T. Cermak, et al. 2010. Targeting DNA double-strand breaks with TAL effector nucleases. Genetics 186(2): 757-61). Cleavage by rare cutting endonucleases usually generates cohesive ends, with 3′ overhangs for LAGLIDADG meganucleases (Chevalier, B. S. and B. L. Stoddard. 2001. Homing endonucleases: structural and functional insight into the catalysts of intron/intein mobility. Nucleic Acids Res 29(18): 3757-74) and 5′ overhangs for Zinc Finger nucleases (Smith, J., M. Bibikova, et al. 2000, Requirements for double-strand cleavage by chimeric restriction enzymes with zinc finger DNA-recognition domains. Nucleic Acids Res 28(17): 3361-9). FokI-based TALE-nucleases (TALENs) have a similar functional layout than Zinc-Finger Nucleases, with the Zinc-finger DNA binding domain being replaced by the TALE domain (Li, T., S. Huang, et al. 2011. TAL nucleases (TALNs): hybrid proteins composed of TAL effectors and FokI DNA-cleavage domain. Nucleic Acids Res 39(1): 359-72; Christian, M., T. Cermak, et al. 2010). Cleavage with Cas endonucleases such as Cas9 endonucleases can result in blunt ends.

An “endonuclease” refers to an enzyme that cleaves the phosphodiester bond within a polynucleotide chain.

Endonucleases include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex.

Type I and Type III restriction endonucleases recognize specific recognition sites, but typically cleave at a variable position from the recognition site, which can be hundreds of base pairs away from the recognition site. In Type II systems the restriction activity is independent of any methylase activity, and cleavage typically occurs at specific sites within or near to the recognition site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, DC).

An “engineered rare-cutting double-strand-break-inducing agent” refers to any rare-cutting double-strand-break-inducing agent that is engineered (modified or derived) from its native form to specifically recognize and induce a double-strand break in the desired recognition site. Thus, an engineered rare-cutting double-strand-break-inducing agent can be derived from a native, naturally-occurring nuclease or it could be artificially created or synthesized. The modification of the nuclease can be as little as one nucleotide. In some embodiments, the engineered rare-cutting double-strand-break-inducing agent induces a double-strand break in a recognition site, wherein the recognition site was not a sequence that would have been recognized by a native (non-engineered or non-modified) rare-cutting double-strand-break-inducing agent. Producing a double-strand break in a recognition site or other DNA can be referred to herein as “cutting” or “cleaving” the recognition site or other DNA.

A “meganuclease” refers to a homing endonuclease, which like restriction endonucleases, bind and cut at a specific recognition site, however the recognition sites for meganucleases are typically longer, about 18 bp or more. In some embodiments of the invention, the meganuclease has been engineered (or modified) to cut a specific endogenous recognition sequence, wherein the endogenous target sequence prior to being cut by the engineered double-strand-break-inducing agent was not a sequence that would have been recognized by a native (non-engineered or non-modified) endonuclease.

A “meganuclease polypeptide” refers to a polypeptide having meganuclease activity and thus capable of producing a double-strand break in the recognition sequence.

Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing open reading frames, introns, and inteins, respectively. For example, intron-, intein-, and freestanding gene encoded meganuclease from Saccharomyces cerevisiae are denoted I-SceI, PI-SceI, and F-SceII, respectively. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-AniI, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp2361P, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-Spomi, I-SpomCP, I-SpomIP, I-SpomIIP, I-SquIP, I-Ssp68031, I-SthPhiJP, I-SthPhiST3P, I-SthPhiSTe3bP, I-TdeIP, I-TevI, I-TevII, I-TeviII, I-UarAP, I-UarHGPAIP, I-UarHGPA13P, I-VinIP, I-ZbiIP, PI-MtuI, PI-MtuHIP PI-MtuHIIP, PI-PfuI, PI-PfuII, PI-PkoI, PI-PkoII, PI-Rma438121P, PI-SpBetaIP, PI-SceI, PI-TfuI, PI-TfuII, PI-ThyI, PI-TliI, PI-TliII, or any active variants or fragments thereof. In a specific embodiment, the engineered endonuclease is derived from I-Cre-I having the sequence set forth in SEQ ID NO: 15, 21 or 26 or an active variant or fragment thereof.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

As used herein, the term “Cas gene” refers to a gene that is generally coupled, associated or close to or in the vicinity of flanking CRISPR loci.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bps, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/024097 published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

The terms “Cas gene”, “CRISPR-associated (Cas) gene” are used interchangeably herein. A comprehensive review of the Cas protein family is presented in Haft et al. (2005) Computational Biology, PLoS Comput Biol 1(6): e60. doi:10.1371/journal.pcbi.0010060. As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

As used herein, the term “Cas endonuclease” refers to a Cas protein encoded by a Cas gene, wherein said Cas protein is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence.

As used herein, the term “guide RNA” refers to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

The term “variable targeting domain” refers to a nucleotide sequence 5-prime of the GUUUU sequence motif in the guide RNA, that is complementary to one strand of a double strand DNA target site in the genome of a plant cell, plant or seed. In one embodiment, the variable targeting domain is 12 to 30 nucleotides in length.

In one embodiment, the guide RNA and Cas endonuclease are capable of forming a complex that enables the Cas endonuclease to introduce a double strand break at a DNA target site.

As used herein, the term “recognition site” refers to a DNA sequence at which a double-strand break is induced in a cell genome by a rare-cutting double-strand-break-inducing agent. The terms “recognition site”, “recognition sequence” are used interchangeably herein. The recognition site can be an endogenous site in a host (such as a yeast or plant) genome, or alternatively, the recognition site can be heterologous to the host (yeast or plant) and thereby not be naturally occurring in the genome, or the recognition site can be found in a heterologous genomic location compared to where it occurs in nature.

As used herein, the term “endogenous recognition site” refers to a rare-cutting double-strand-break-inducing agent recognition site that is endogenous or native to the genome of a host (such as a plant or yeast) and is located at the endogenous or native position of that recognition site in the genome of the host (such as a plant or yeast). The length of the recognition site can vary, and includes, for example, recognition sites that are at least 4, 6, 8, 10, 12, 14, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length. It is further possible that the recognition site could be palindromic, that is, the sequence on one strand reads the same in the opposite direction on the complementary strand. The nick/cleavage site could be within the recognition sequence or the nick/cleavage site could be outside of the recognition sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

As used herein, the term “intended recognition site” refers to the recognition sequence to which an engineered rare-cutting double-strand-break-inducing agent, such as an engineered meganuclease, was directed to specifically recognize and induce a double-strand break. In one embodiment, the rare-cutting double-strand-break-inducing agent is a LIG3-4 engineered meganuclease (SEQ ID NO: 2) which was designed to recognize the intended recognition sequence of SEQ ID NO: 13 (US patent publication 2009-0133152 A1, published May 21, 2009). In another embodiment, the rare-cutting double-strand-break-inducing agent is a MHP14+ engineered meganuclease (SEQ ID NO: 4) which was designed to recognize the intended recognition sequence of SEQ ID NO: 14 (in U.S. patent application Ser. No. 13/427,138 filed on Mar. 22, 2012).

As used herein, the term “variant recognition site” refers to a variant nucleotide sequence that comprises at least one base nucleotide alteration when compared to the intended recognition site to which an engineered rare-cutting double-strand-break-inducing agent such as a meganuclease, was directed to specifically recognize and induce a double-strand break. Such “alteration” include, for example: (i) replacement of at least one nucleotide, (ii) a deletion of at least one nucleotide, (iii) an insertion of at least one nucleotide, or (iv) any combination of (i)-(iii). Active variants and fragments of the recognition can comprise at least 65%, 70%, 75%, 80%, 81%, 82%, 83%, 84%, 85%, 86%, 87%, 88%. 89% 90%, 91%, 92%, 93%, 94%, 95%, 96%, 97%, 98%, 99% or more sequence identity to the given recognition sequence, wherein the active variants retain biological activity and hence are capable of being recognized and cleaved by an endonuclease. Variant recognition sites can comprise at least one (1) and up to 2, 3, 5, 6, 7, 8, 9, 10, 11, 12, 13, 14, 15, 16, 17, 18, 19 or 20 off-nucleotide preferences. In one embodiment, variant recognition sites are non-endogenous to the host genome, such variant recognition sites include, but are not limited to, the maize variant recognition sites shown in FIG. 4 (SEQ ID NOs: 15-22 and SEQ ID NOs: 23-35). In another embodiment, the variant recognition sites are present in the host genome (referred to as genomic variant recognition sites) or endogenous to the host genome, such as plant or yeast genomes. In some embodiments, the variant recognition sites can be introduced into a plant genome by the mutagenesis of an endogenous genomic sequence. Methods for the site-specific mutagenesis of genomic DNA are known in the art, and include those described, for example in U.S. Pat. Nos. 5,565,350, 5,731,181, and 6,870,075. Other methods include the use of zinc finger nucleases, such as those methods described in U.S. Patent Publication 20050208489.

A “genomic variant recognition site” refers to a variant recognition site of a rare-cutting double-strand-break-inducing agent, such as a meganuclease, that is endogenous to the genome of an organism (such as a plant or yeast). One example of a variant recognition site that is endogenous to the maize genome is SEQ ID NO: 11.

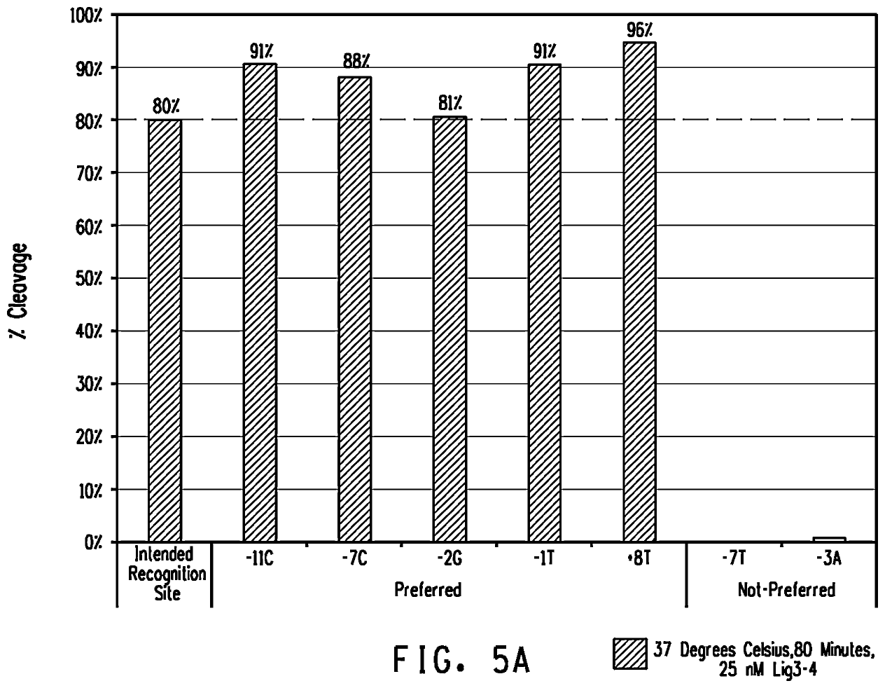

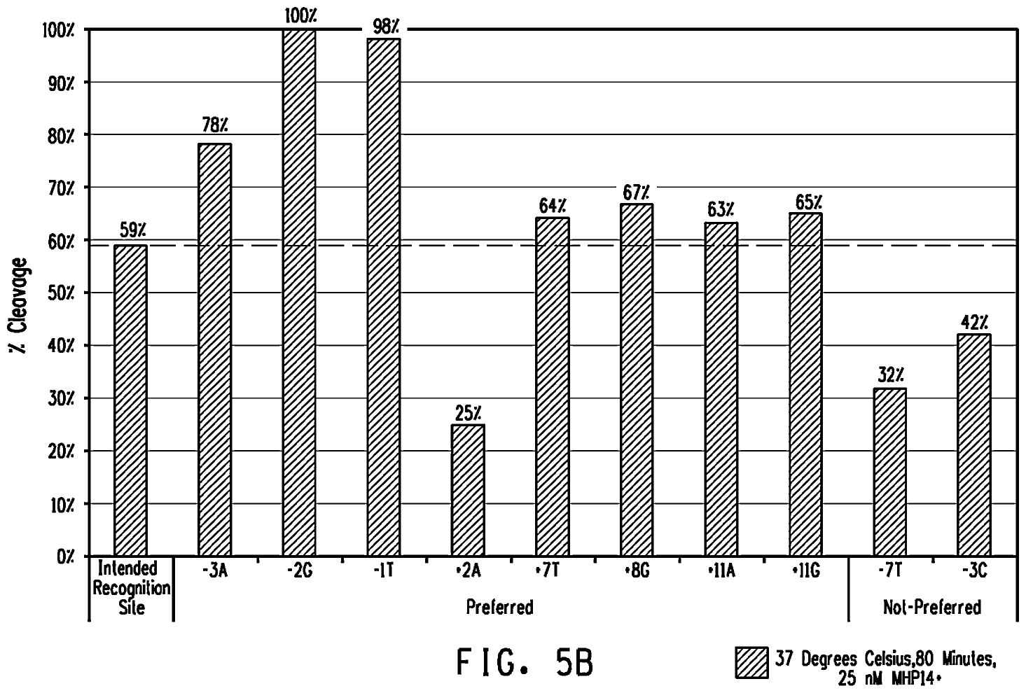

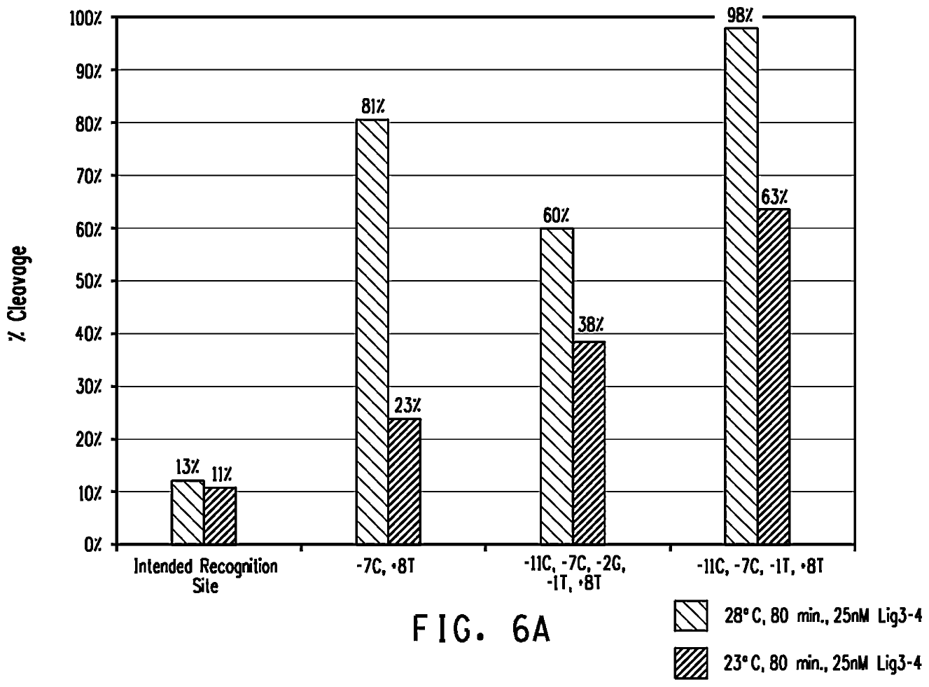

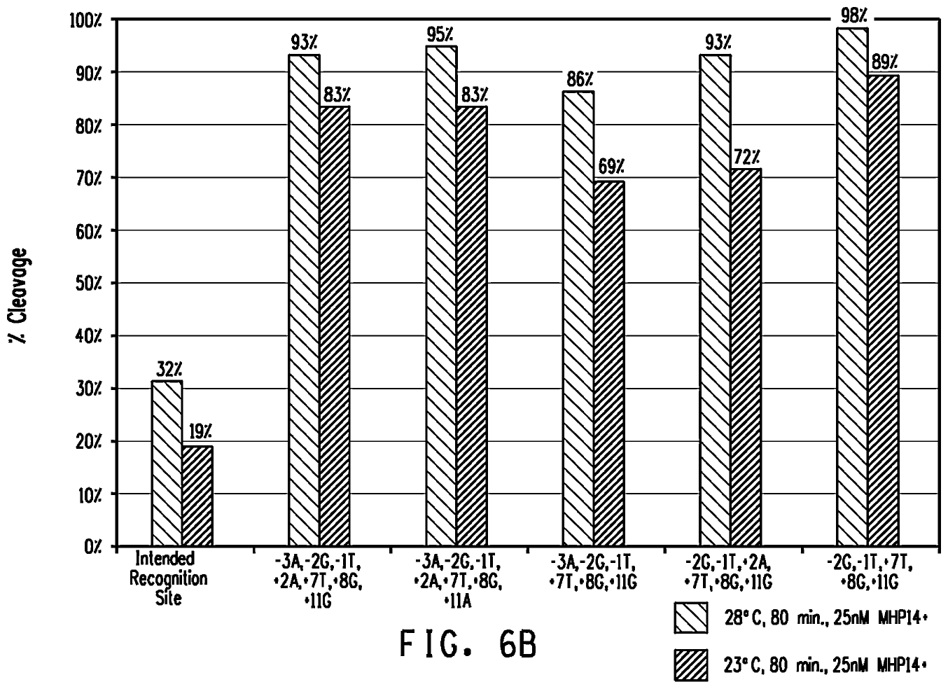

The term “preferred off-nucleotides” or “off-nucleotide preferences” can be used interchangeably and refers to nucleotides that are located at the same position relative to the nucleotides of the intended recognition site, but are more prevalent in the identified genomic variant recognition sites (see for example the prevalence for a +8T (80%) compared with an intended recognition site+8C (13%) in FIG. 3A). In most instances, the preferred off-nucleotide when placed into the intended recognition site is cleaved at a higher percentage than the intended recognition site (see for example +8T (96% cleavage) compared to +8C (80% cleavage in FIG. 5A).

In one embodiment, the intended recognition sequence of the LIG3-4 engineered meganuclease comprises SEQ ID NO: 13, whereas the variant recognition site of the LIG3-4 engineered meganuclease comprises SEQ ID NO: 15, 16, 17, 18, 19, 20, 21, or 22. In another embodiment, the intended recognition sequence of the MHP14+ engineered meganuclease comprises SEQ ID NO: 14, whereas the variant recognition site of the MHP14+ engineered meganuclease comprises SEQ ID NO: 23, 24, 25, 26, 27, 28, 29, 30, 31, 32, 33, 34, or 35.

A “variant recognition site locus” is the position on a chromosome comprising the variable recognition site. Preferably, the variant recognition site locus is within 0, 50, 100, 200, 300, 400, 500, 600, 700, 800, 900 or 1000 base pairs of the variant recognition site.

The term “meganuclease activity” as used herein refers to the ability of a meganuclease to produce a double-strand break at a desired recognition sequence and thus retain double-strand-break-inducing activity. Producing the double-strand break in a recognition sequence or other DNA can be referred to herein as “cutting” or “cleaving” the recognition sequence or other DNA.

Assays for meganuclease activity are known and generally measure the overall activity and specificity of the meganuclease on DNA substrates containing the recognition site. These DNA substrates include but are not limited to genomic DNA and plasmid DNA. For example the meganuclease activity can be measured in-vitro as described herein in Example 3 and Example 9. In short, time-course digestions can be carried out at 37° C., 28° C., and 23° C. (or any temperature ranging between 37° C., 36° C., 35° C., 34° C., 33° C., 32° C., 31° C., 30° C., 29° C., 28° C., 27° C., 26° C., 25° C., 24° C. and 23° C.), on plasmid or genomic DNA containing a meganuclease recognition site and the % digestion of each sample (also referred to as % cleavage or to as % loss of meganuclease recognition sites) (indicative of meganuclease activity) can be determined by real-time PCR.

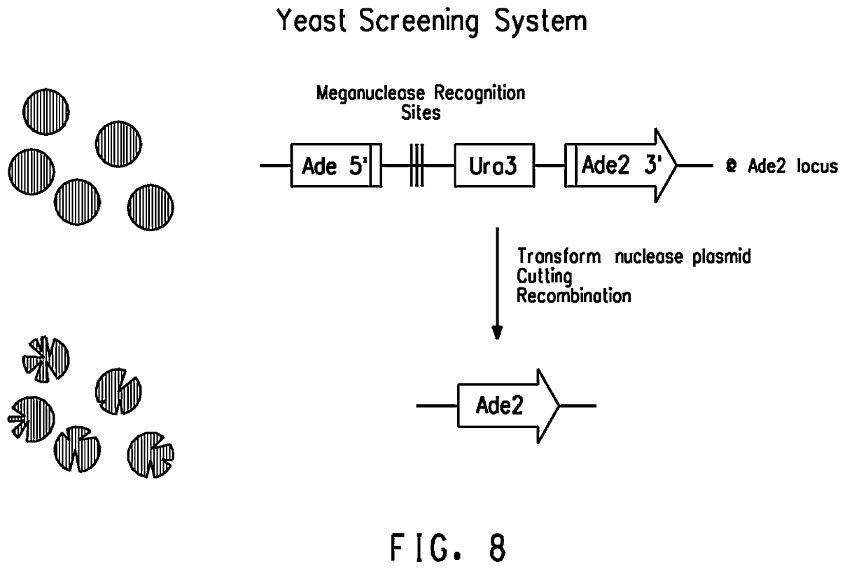

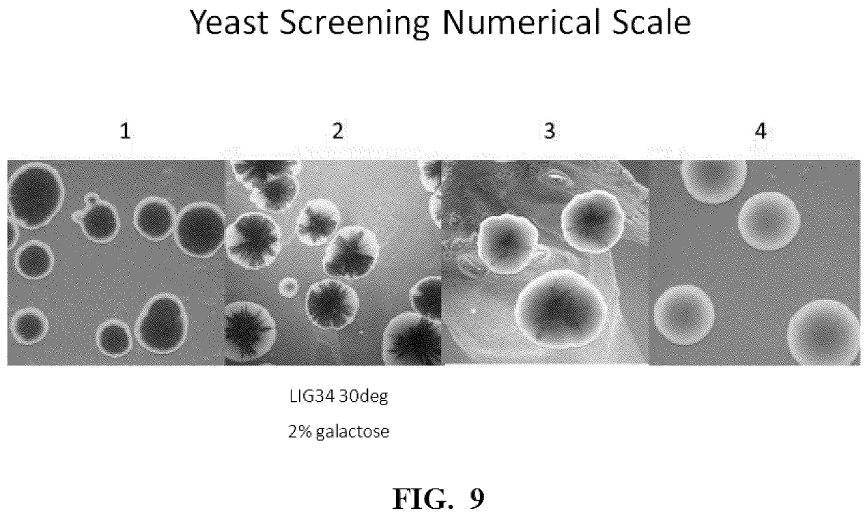

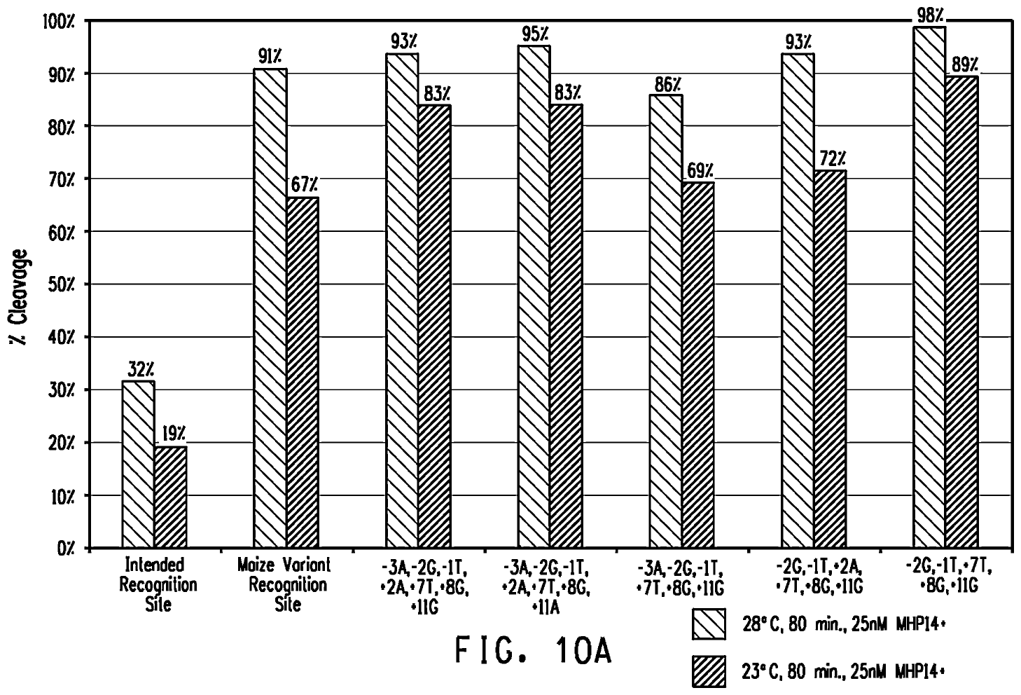

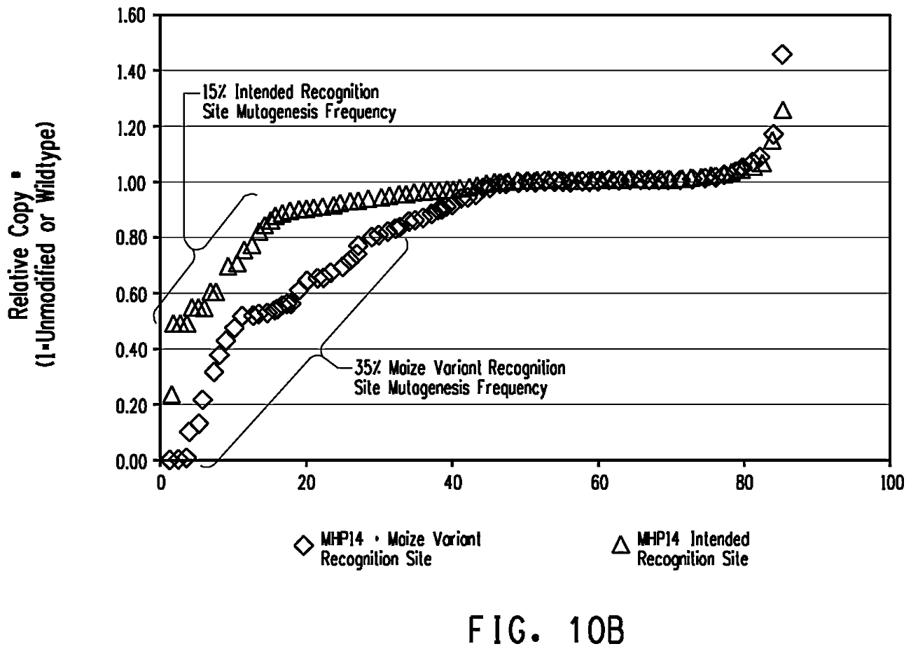

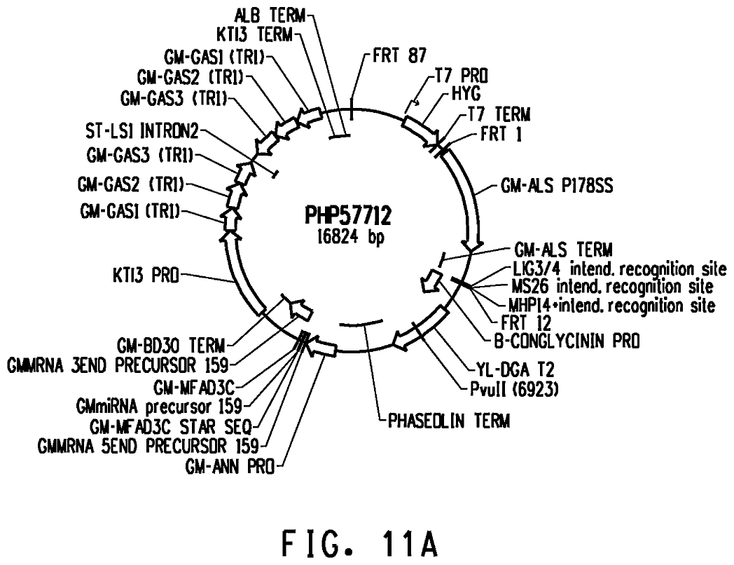

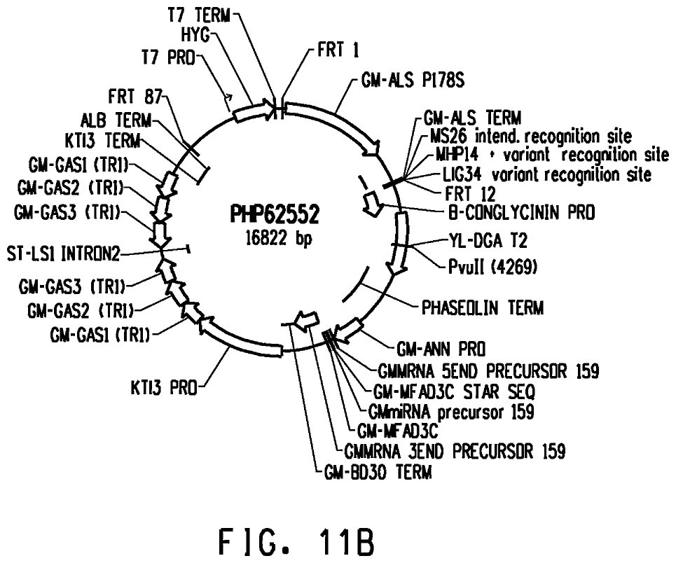

Meganuclease activity can also be measured using a yeast screening assay as described herein (FIGS. 8 and 9 and Example 16). In short, yeast cells with a functional Ade2 gene are white, whereas those lacking Ade2 function exhibit red pigmentation due to accumulation of a metabolite earlier in the adenine biosynthetic pathway resulting in red colonies with white sectors. The degree of white sectoring, sometimes extending to entire colonies, indicates the amount of meganuclease cutting activity. Since the sectoring phenotype is a qualitative measure of meganuclease activity, a 0-4 numerical scoring system was implemented. As shown in FIG. 3, a score of 0 indicates that no white sectors (no meganuclease cutting) were observed; a score of 4 indicates completely white colonies (complete cutting of the recognition site); scores of 1-3 indicate intermediate white sectoring phenotypes (and intermediate degrees of recognition site cutting.

Furthermore, meganuclease activity can be measured in-planta by determining the Target Site (TS) mutation rate. Target site mutation rate is defined as: (number of events with target site modification/total number events)*100%.

An “increased” or an “increased” activity are used interchangeably herein. An “increased” or “increased” meganuclease activity comprises any statistically significant increase in the activity of the parental meganuclease polypeptide as determined through any activity assays described herein.

The meganuclease can be provided via a polynucleotide encoding the endonuclease. Such a polynucleotide encoding an endonuclease can be modified to substitute codons having a higher frequency of usage in a plant, as compared to the naturally occurring polynucleotide sequence. For example the polynucleotide encoding the meganuclease can be modified to substitute codons having a higher frequency of usage in a maize or soybean plant, as compared to the naturally occurring polynucleotide sequence.

A “control meganuclease” or “reference meganuclease” can be used interchangeably and refers to any meganuclease to which a variant meganuclease is compared to. Control meganucleases can include, but are not limited to, parental or corresponding meganucleases or any wild-type I-CreI type meganucleases.

Numbering of an amino acid or nucleotide polymer, such any one of the meganucleases of the invention, corresponds to numbering of a selected amino acid polymer or nucleic acid when the position of a given monomer component (amino acid residue, incorporated nucleotide, etc.) of the polymer corresponds to the same residue position in a selected reference polypeptide or polynucleotide.

As used herein, a “genomic region of interest” is a segment of a chromosome in the genome of a plant that is desirable for introducing a polynucleotide of interest or trait of interest. The genomic region of interest can include, for example, one or more polynucleotides of interest. Generally, a genomic region of interest of the present invention comprises a segment of chromosome that is 0-15 centi-morgan (cM).

As used herein, a “polynucleotide of interest” within a genomic region of interest is any coding and/or non-coding portion of the genomic region of interest including, but not limited to, a transgene, a native gene, a mutated gene, and a genetic marker such as, for example, a single nucleotide polymorphism (SNP) marker and a simple sequence repeat (SSR) marker.

As used herein, “physically linked,” “in physical linkage”, and “genetically linked” are used to refer to any two or more genes, transgenes, native genes, mutated genes, alterations, target sites, markers, and the like that are part of the same DNA molecule or chromosome.

As used herein, an “isolated” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein. When the polypeptide of the invention or biologically active portion thereof is recombinantly produced, optimally culture medium represents less than about 30%, 20%, 10%, 5%, or 1% (by dry weight) of chemical precursors or non-protein-of-interest chemicals.

As used herein, polynucleotide or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene is recombinant.

A “subsequence” or “fragment” is any portion of an entire sequence.

### Sequence Comparisons

The following terms are used to describe the sequence relationships between two or more polynucleotides or polypeptides: (a) “reference sequence”, (b) “comparison window”, (c) “sequence identity”, and, (d) “percent sequence identity.”

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence or protein sequence.

As used herein, “comparison window” makes reference to a contiguous and specified segment of a polypeptide sequence, wherein the polypeptide sequence in the comparison window may comprise additions or deletions (i.e., gaps) compared to the reference sequence (which does not comprise additions or deletions) for optimal alignment of the two polypeptides. Generally, the comparison window is at least 5, 10, 15, or 20 contiguous amino acid in length, or it can be 30, 40, 50, 100, or longer. Those of skill in the art understand that to avoid a high similarity to a reference sequence due to inclusion of gaps in the polypeptide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. BLASTP protein searches can be performed using default parameters. See, blast.ncbi.nlm.nih.gov/Blast.cgi.

Sequence alignments and percent similarity calculations may be determined using the Megalign program of the LASARGENE bioinformatics computing suite (DNASTAR Inc., Madison, WI) or using the AlignX program of the Vector NTI bioinformatics computing suite (Invitrogen, Carlsbad, CA). Multiple alignment of the sequences are performed using the Clustal method of alignment (Higgins and Sharp, CABIOS 5:151-153 (1989)) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are GAP PENALTY=10, GAP LENGTH PENALTY=10, KTUPLE=2, GAP PENALTY=5, WINDOW=4 and DIAGONALS SAVED=4. A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN refers to a BLAST program that compares a nucleotide query sequence against a nucleotide sequence database.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for increased expression in a host cell, it is desirable to design the nucleic acid fragment such that its frequency of codon usage approaches the frequency of preferred codon usage of the host cell.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percent sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

### Polynucleotide Constructs

Provided herein are polynucleotides or nucleic acid molecules comprising the variant recognition sites for rare-cutting double-strand-break-inducing agents or any active variants or fragments thereof. The terms “polynucleotide,” “polynucleotide sequence,” “nucleic acid sequence,” and “nucleic acid fragment” are used interchangeably herein. These terms encompass nucleotide sequences and the like. The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

Further provided are recombinant polynucleotides comprising the various rare-cutting double-strand-break-inducing agents such as engineered meganucleases. The terms “recombinant polynucleotide”, “recombinant nucleotide”, “recombinant DNA” and “recombinant DNA construct” are used interchangeably herein. A recombinant construct comprises an artificial or heterologous combination of nucleic acid sequences, e.g., regulatory and coding sequences that are not found together in nature. For example, a transfer cassette can comprise restriction sites and a heterologous polynucleotide of interest. In other embodiments, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments provided herein. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The meganuclease polynucleotides disclosed herein can be provided in expression cassettes for expression in the plant of interest. The cassette can include 5′ and 3′ regulatory sequences operably linked to a meganuclease polynucleotide or active variant or fragment thereof. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the meganuclease polynucleotide or active variant or fragment thereof to be under the transcriptional regulation of the regulatory regions. The expression cassette may additionally contain selectable marker genes.

The expression cassette can include in the 5-3′ direction of transcription, a transcriptional and translational initiation region (i.e., a promoter), a meganuclease polynucleotide or active variant or fragment thereof, and a transcriptional and translational termination region (i.e., termination region) functional in plants. The regulatory regions (i.e., promoters, transcriptional regulatory regions, and translational termination regions) and/or the meganuclease polynucleotide or active variant or fragment thereof may be native/analogous to the host cell or to each other. Alternatively, the regulatory regions and/or the meganuclease polynucleotide of or active variant or fragment thereof may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

While it may be optimal to express the sequences using heterologous promoters, the native promoter sequences may be used. Such constructs can change expression levels of the meganuclease polynucleotide in the plant or plant cell. Thus, the phenotype of the plant or plant cell can be altered.

The termination region may be native with the transcriptional initiation region, may be native with the operably linked meganuclease polynucleotide or active variant or fragment thereof, may be native with the plant host, or may be derived from another source (i.e., foreign or heterologous) to the promoter, the meganuclease polynucleotide or active fragment or variant thereof, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385. See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used to express the various meganuclease sequence disclosed herein, including the native promoter of the polynucleotide sequence of interest. The promoters can be selected based on the desired outcome. Such promoters include, for example, constitutive, tissue-preferred, or other promoters for expression in plants.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced meganuclease expression within a particular plant tissue. Tissue-preferred promoters include those described in Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Synthetic promoters can be used to express meganuclease sequences or biologically active variants and fragments thereof.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, sulfonylureas, dicamba, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the present invention.

### Method of Introducing

The rare-cutting double-strand-break-inducing agent, such as a meganuclease may be introduced by any means known in the art. For example, a cell, yeast or plant having the intended or variant recognition site in its genome is provided. The meganuclease may be transiently expressed or the polypeptide itself can be directly provided to the cell. Alternatively, a nucleotide sequence capable of expressing the meganuclease may be stably integrated into the genome of the plant. In the presence of the corresponding intended or variant recognition site and the meganuclease, a donor DNA can be inserted into the transformed plant's genome. Alternatively, the different components may be brought together by sexually crossing transformed plants. Thus a sequence encoding a meganuclease and/or intended or variant recognition site can be sexually crossed to one another to allow each component of the system to be present in a single plant. The meganuclease may be under the control of a constitutive or inducible promoter. Such promoters of interest are discussed in further detail elsewhere herein.

Various methods can be used to introduce a sequence of interest such as, any of the rare-cutting double-strand-break-inducing agents into a plant or plant part. “Introducing” is intended to mean presenting to the plant, plant cell or plant part the polynucleotide or polypeptide in such a manner that the sequence gains access to the interior of a cell of the plant. The methods of the invention do not depend on a particular method for introducing a sequence into a plant or plant part, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the rare-cutting double-strand-break-inducing agent sequence, such as a meganuclease sequence, or active variant or fragments thereof can be provided to a yeast cell or plant using a variety of transient transformation methods. Such transient transformation methods include, but are not limited to, the introduction of the meganuclease protein or active variants and fragments thereof directly into a yeast cell or plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

Generally, such methods involve incorporating a nucleotide construct of the invention within a DNA or RNA molecule. It is recognized that the an meganuclease sequence may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

As used herein, “primers” are isolated polynucleotides that are annealed to a complementary target DNA strand by nucleic acid hybridization to form a hybrid between the primer and the target DNA strand, then extended along the target DNA strand by a polymerase, e.g., a DNA polymerase. Primer pairs of the invention refer to their use for amplification of a target polynucleotide, e.g., by the polymerase chain reaction (PCR) or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference.

Probes and primers are of sufficient nucleotide length to bind to the target DNA sequence and specifically detect and/or identify a polynucleotide encoding a meganuclease polypeptide or active variant or fragment thereof as describe elsewhere herein. It is recognized that the hybridization conditions or reaction conditions can be determined by the operator to achieve this result. This length may be of any length that is of sufficient length to be useful in a detection method of choice. Such probes and primers can hybridize specifically to a target sequence under high stringency hybridization conditions. Probes and primers according to embodiments of the present invention may have complete DNA sequence identity of contiguous nucleotides with the target sequence, although probes differing from the target DNA sequence and that retain the ability to specifically detect and/or identify a target DNA sequence may be designed by conventional methods. Accordingly, probes and primers can share about 80%, 85%, 90%, 91%, 92%, 93%, 94%, 95%, 96%, 97%, 98%, 99% or greater sequence identity or complementarity to the target polynucleotide.

Methods for preparing and using probes and primers are described, for example, in Molecular Cloning: A Laboratory Manual, 2.sup.nd ed, vol. 1-3, ed. Sambrook et al., Cold Spring Harbor Laboratory Press, Cold Spring Harbor, N.Y. 1989 (hereinafter, “Sambrook et al., 1989”); Current Protocols in Molecular Biology, ed. Ausubel et al., Greene Publishing and Wiley-Interscience, New York, 1992 (with periodic updates) (hereinafter, “Ausubel et al., 1992”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 10 (Invitrogen); PrimerSelect (DNASTAR Inc., Madison, Wis.); and Primer (Version 0.5.COPYRGT., 1991, Whitehead Institute for Biomedical Research, Cambridge, Mass.). Additionally, the sequence can be visually scanned and primers manually identified using guidelines known to one of skill in the art.

### Yeast and Plants

Yeast, plants, plant cells, plant parts and seeds, and grain having the variant recognition sequences for rare-cutting double-strand-break-inducing agents, such as meganucleases disclosed herein, are provided. In specific embodiments, the yeast, plants and/or plant parts have stably incorporated at least one heterologous variant recognition sequence disclosed herein or an active variant or fragment thereof. Thus, yeast, plants, plant cells, plant parts and seed are provided which comprise at least one variant recognition sequence of any one of SEQ ID NOs: 15, 16, 17, 18, 19, 20, 21, 22, 23, 24, 25, 26, 27, 28, 29, 30, 31, 32, 33, 34, 35 or any combination thereof, or a biologically active fragment and/or variant thereof. In specific embodiments, the variant recognition sequences expresses increased cleavage activity towards the rare-cutting double-strand-break-inducing agent.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

A transformed plant or transformed plant cell provided herein is one in which genetic alteration, such as transformation, has been affected as to a gene of interest, or is a plant or plant cell which is descended from a plant or cell so altered and which comprises the alteration. A “transgene” is a gene that has been introduced into the genome by a transformation procedure. Accordingly, a “transgenic plant” is a plant that contains a transgene, whether the transgene was introduced into that particular plant by transformation or by breeding; thus, descendants of an originally-transformed plant are encompassed by the definition. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which does not express the transgene, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the transgene; or (e) the subject plant or plant cell itself, under conditions in which the construct is not expressed.

Plant cells that have been transformed to express a meganuclease provided herein can be grown into whole plants. The regeneration, development, and cultivation of plants from single plant protoplast transformants or from various transformed explants is well known in the art. See, for example, McCormick et al. (1986) Plant Cell Reports 5:81-84; Weissbach and Weissbach, In: Methods for Plant Molecular Biology, (Eds.), Academic Press, Inc. San Diego, Calif., (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. Two or more generations may be grown to ensure that expression of the desired phenotypic characteristic is stably maintained and inherited and then seeds harvested to ensure expression of the desired phenotypic characteristic has been achieved. In this manner, the compositions presented herein provide transformed seed (also referred to as “transgenic seed”) having a polynucleotide provided herein, for example, a target site, stably incorporated into their genome.

The variant recognition sequences and active variant and fragments thereof disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus. In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Non-limiting examples of compositions and methods disclosed herein are as follows:
1) A method to identify a variant recognition site for a rare-cutting engineered double-strand-break-inducing agent capable of introducing a rare double strand break in an intended recognition site, said method comprising:
a) contacting genomic DNA with a rare-cutting engineered double-strand-break-inducing agent capable of introducing a double-strand break into said genomic DNA, wherein the double-strand break results in a nucleotide overhang;
b) ligating a first adapter to said nucleotide overhang;
c) shearing the ligated DNA obtained in step (b) and ligating at least one second adapter to the sheared nucleotide end to allow for the amplification and sequencing of genomic DNA fragments surrounding the double strand break;
d) aligning nucleotide sequences of the DNA fragments obtained in (c) with a reference genome DNA sequence; and,
e) identifying a variant recognition site comprising at least one nucleotide base alteration when compared to the intended recognition site of said engineered double-strand break-inducing agent.
2) The method of embodiment 1 wherein the rare-cutting engineered double-strand-break-inducing agent is selected from the group consisting of a meganuclease, a zinc finger nuclease, a TAL effector nuclease, a transposase, and a site-specific recombinase.
3) The method of embodiment 1 wherein the nucleotide overhang is a 3′ nucleotide overhang.
4) The method of embodiment 1 wherein the nucleotide overhang is a 5′ nucleotide overhang.
5) The method of embodiment 1 wherein the first adapter ligated to the nucleotide overhang is a non-5′ phosphorylated adapter.
6) The method of embodiment 1 wherein the genomic DNA is selected from the group consisting of a prokaryotic DNA, eukaryotic DNA and synthetic DNA.
7) The method of embodiment 6 wherein the eukaryotic DNA is isolated from a plant, yeast or animal.
8) The method of embodiment 7 wherein the plant is selected from the group consisting of soybean, sunflower, cotton, alfalfa, canola, cotton, tobacco, potato, Arabidopsis, safflower, maize, rice, sorghum, barley, wheat, millet, oats, sugarcane, turfgrass, and switch grass.
9) The method of embodiment 1, wherein the double-strand-break-inducing agent is derived from I-CreI.
10) A method to identify a variant recognition site with an improved cleavage activity for a rare-cutting engineered double-strand-break-inducing agent capable of introducing a rare double strand break in an intended recognition site, said method comprising:
a) contacting genomic DNA with rare-cutting engineered double-strand-break-inducing agent capable of introducing a double strand break into said genomic DNA, wherein the double strand break results in a nucleotide overhang;
b) ligating a first adapter to said nucleotide overhang;
c) shearing the ligated DNA obtained in step (b) and ligating a second adapter to the sheared nucleotide end to allow for the amplification and sequencing of genomic DNA fragments surrounding the double strand break;
d) aligning nucleotide sequences of the DNA fragments obtained in (c) with a reference genome DNA sequence; and,
e) identifying a variant recognition site comprising at least one nucleotide base alteration when compared to the intended recognition site of said rare-cutting engineered double-strand-break-inducing agent;
f) analyzing the a rare-cutting engineered double-strand-break-inducing agent activity at the variant recognition sites of d);
g) identifying a variant recognition site that results in an increased activity of the rare-cutting engineered double-strand-break-inducing agent when compared to the activity at the intended recognition site.
11) The method of embodiment 10 wherein the increased activity of the rare-cutting engineered double-strand-break-inducing agent is evidenced by
a) a higher percent (%) cleavage of the variant recognition site when compared to the percent (%) cleavage of intended recognition site, wherein the recognition sites are located on genomic DNA;
b) a higher percent (%) cleavage of the variant recognition site when compared to the percent (%) cleavage of intended recognition site, wherein the recognition sites are located on plasmid DNA;
c) a higher yeast assay score for the variant recognition site when compared to the intended recognition site; or,
d) any combination of (a), (b) and (c)
12) A method for introducing into the genome of a cell a variant recognition site for a rare-cutting engineered double-strand-break-inducing agent capable of introducing a rare double strand break in an intended recognition site, said method comprising:
a) providing a donor DNA comprising a variant recognition site for a rare-cutting engineered double-strand-break-inducing agent capable of introducing a double-strand break in an intended recognition site, wherein said a rare-cutting engineered double-strand-break-inducing agent is also capable of introducing a double-strand break in said variant recognition site;
b) providing a plant cell;
c) contacting the plant cell with the donor DNA; and,

d) identifying at least one plant cell from (c) comprising in its genome said variant recognition site.
13) The method of embodiment 12 wherein the rare-cutting engineered double-strand-break-inducing agent is selected from the group consisting of a meganuclease, a zinc finger nuclease, a TAL effector nuclease, a transposase, a Cas endonuclease and a site-specific recombinase.
14) An isolated polynucleotide comprising a variant recognition site with an improved cleavage activity for an engineered meganuclease capable of introducing a double strand break in an intended recognition site, wherein said variant recognition site comprises a nucleotide sequence with at least 1 nucleotide base substitution when compared to the intended recognition site of SEQ ID NO: 14.
15) The isolated polynucleotide of embodiment 1, wherein said variant recognition site comprises a sequence with at least 2, 3, 4, 5, 6 or 7 base pair alterations when compared to SEQ ID NO: 14.
16) The isolated polynucleotide of embodiment 14, wherein said variant recognition site comprises:
a) an adenine (A) at a position corresponding to the nucleotide position 9 in SEQ ID NO: 14;
b) a guanine (G) at a position corresponding to the nucleotide position 10 in SEQ ID NO: 14;
c) a thymine (T) at a position corresponding to the nucleotide position 11 in SEQ ID NO: 14;
d) an adenine (A) at a position corresponding to the nucleotide position 13 in SEQ ID NO: 14;
e) a thymine (T) at a position corresponding to the nucleotide position 18 in SEQ ID NO: 14;
f) a guanine (G) at a position corresponding to the nucleotide position 19 in SEQ ID NO: 14;
g) a guanine (G) or an adenine (A) at a position corresponding to the nucleotide position 22 in SEQ ID NO: 14; or,
h) any combination of a) to g).
17) The isolated polynucleotide of embodiment 14, wherein said variant recognition sequence is selected from the group consisting of SEQ ID NOs: 23, 24, 25, 26, 27, 28, 29, 30, 31, 32, 33, 34 and 35.
18) The isolated polynucleotide of embodiment 14, wherein the improved cleavage activity is evidenced by:
a) a higher percent (%) cleavage of the variant recognition site when compared to the percent (%) cleavage of intended recognition site of SEQ ID NO:14, wherein the recognition sites are located on genomic DNA;
b) a higher percent (%) cleavage of the variant recognition site when compared to the percent (%) cleavage of intended recognition site, wherein the recognition sites are located on plasmid DNA;
c) a higher yeast assay score for the variant recognition site when compared to the intended recognition site; or, d) any combination of (a), (b) and (c)
19) A recombinant DNA fragment comprising the isolated polynucleotide of embodiment 14.
20) A cell comprising the recombinant DNA fragment of embodiment 19.
21) The cell of embodiment 20, wherein the cell is a yeast or plant cell.
22) A transgenic plant or seed comprising the plant cell of embodiment 21.
23) The transgenic plant of embodiment 22 wherein said plant is selected from the group consisting of maize, wheat, rice, barley, sugarcane, sorghum, rye, switch grass, soybean, Brassica, sunflower, cotton, or alfalfa.
24) An isolated polynucleotide comprising a variant recognition site with an improved cleavage activity for an engineered meganuclease capable of introducing a double strand break in an intended recognition site, wherein said variant recognition site comprises a nucleotide sequence with at least 1 nucleotide base substitution when compared to the intended recognition site of SEQ ID NO: 13.
25) The isolated polynucleotide of embodiment 24, wherein said variant recognition site comprises a sequence with at least 2, 3, 4, or 5 base pair alterations when compared to SEQ ID NO: 13.
26) The isolated polynucleotide of embodiment 24, wherein said variant recognition site comprises:
a) a cytosine (C) at a position corresponding to the nucleotide position 1 in SEQ ID NO: 13
b) a cytosine (C)) at a position corresponding to the nucleotide position 5 in SEQ ID NO: 13;
c) a guanine (G) at a position corresponding to the nucleotide position 10 in SEQ ID NO: 13;
d) a thymine (T) an adenine (A) at a position corresponding to the nucleotide position 11 in SEQ ID NO: 13;
e) a thymine (T) at a position corresponding to the nucleotide position 19 in SEQ ID NO: 13;
f) any combination of a) to e).
27) The isolated polynucleotide of embodiment 24, wherein said variant recognition sequence is selected from the group consisting of SEQ ID NOs: 15, 16, 17, 18, 19, 20 and 21.
28) The isolated polynucleotide of embodiment 24, wherein the improved cleavage activity is evidenced by:
a) a higher percent (%) cleavage of the variant recognition site when compared to the percent (%) cleavage of intended recognition site of SEQ ID NO:13, wherein the recognition sites are located on genomic DNA;
b) a higher percent (%) cleavage of the variant recognition site when compared to the percent (%) cleavage of intended recognition site, wherein the recognition sites are located on plasmid DNA;
c) a higher yeast assay score for the variant recognition site when compared to the intended recognition site; or,
d) any combination of (a), (b) and (c).
29) A recombinant DNA fragment comprising the isolated polynucleotide of embodiment 24.
30) A cell comprising the recombinant DNA fragment of embodiment 29.
31) The cell of embodiment 30, wherein the cell is a yeast or plant cell.
32) A transgenic plant or seed comprising the plant cell of embodiment 31.
33) The transgenic plant of embodiment 32 wherein said plant is selected from the group consisting of maize, wheat, rice, barley, sugarcane, sorghum, rye, switch grass, soybean, Brassica, sunflower, cotton, or alfalfa.
34) A method for targeting the insertion of a polynucleotide of interest to a specific chromosomal site within a plant genome, said method comprising:
a) transforming a plant cell or a plant with a DNA fragment comprising a polynucleotide of interest, wherein said genome of said plant cell or plant comprises at least one variant recognition site selected from the group consisting of SEQ ID NOs: 15, 16, 17, 18, 19, 20 and 21; and,
b) providing a meganuclease capable of providing a double strand break into the variable recognition site of (a); and,
c) selecting said plant cell or plant comprising said polynucleotide of interest integrated into said variant recognition site.
35) The method of embodiment 34 wherein providing said meganuclease comprises integrating in the genome of said plant cell or plant a nucleotide sequence encoding the meganuclease of SEQ ID NO: 1.
36) A plant or plant cell obtained by the method of embodiment 34.
37) A method for targeting the insertion of a polynucleotide of interest to a specific chromosomal site within a plant genome, said method comprising:
a) transforming a plant cell or a plant with a DNA fragment comprising a polynucleotide of interest, wherein said genome of said plant cell or plant comprises at least one variant recognition site selected from the group consisting of SEQ ID NOs: 23, 24, 25, 26, 27, 28, 29, 30, 31, 32, 33, 34 and 35;
b) providing a meganuclease capable of providing a double strand break into the variable recognition site of (a); and
c) selecting said plant cell or plant comprising said polynucleotide of interest integrated into said variant recognition site.
38) The method of embodiment 37 wherein providing the meganuclease comprises integrating in the genome of said plant cells a nucleotide sequence encoding the meganuclease of SEQ ID NO: 3
39) A plant or plant cell obtained by the method of embodiment 37.
40) A method to identify a variant recognition site for a rare-cutting engineered double-strand-break-inducing agent capable of introducing a rare double strand break in an intended recognition site, said method comprising:
a) contacting genomic DNA with a rare-cutting engineered double-strand-break-inducing agent capable of introducing a double-strand break into said genomic DNA, wherein the double-strand break results in a blunt end;
b) creating a nucleotide overhang from the blunt end of (a);
c) ligating a first adapter to the nucleotide overhang of (b);
d) shearing the ligated DNA obtained in step (c) and ligating at least one second adapter to the sheared nucleotide end to allow for the amplification and sequencing of genomic DNA fragments surrounding the double strand break;
e) aligning nucleotide sequences of the DNA fragments obtained in (d) with a reference genome DNA sequence; and,
f) identifying a variant recognition site comprising at least one nucleotide base alteration when compared to the intended recognition site of said engineered double-strand break-inducing agent.
41) The method of embodiment 40 wherein the rare-cutting engineered double-strand-break-inducing agent is a Cas endonuclease
42) The method of embodiment 41 wherein the Cas endonuclease is capable of forming a complex with a guide RNA, wherein said complex enables the Cas endonuclease to introduce a double strand break into said genomic DNA.
43) The method of embodiment 40 wherein the genomic DNA is selected from the group consisting of a prokaryotic DNA, eukaryotic DNA and synthetic DNA.
44) The method of embodiment 43 wherein the eukaryotic DNA is isolated from a plant, yeast or animal.
45) A method to identify a variant recognition site with an improved cleavage activity for a rare-cutting engineered double-strand-break-inducing agent capable of introducing a rare double strand break in an intended recognition site, said method comprising:
a) contacting genomic DNA with rare-cutting engineered double-strand-break-inducing agent capable of introducing a double strand break into said genomic DNA, wherein the double strand break results in a blunt end
b) creating a nucleotide overhang from the blunt end of (a);
c) ligating a first adapter the nucleotide overhang of (b);
d) shearing the ligated DNA obtained in step (c) and ligating a second adapter to the sheared nucleotide end to allow for the amplification and sequencing of genomic DNA fragments surrounding the double strand break;
e) aligning nucleotide sequences of the DNA fragments obtained in (d) with a reference genome DNA sequence; and,
f) identifying a variant recognition site comprising at least one nucleotide base alteration when compared to the intended recognition site of said rare-cutting engineered double-strand-break-inducing agent;
g) analyzing the rare-cutting engineered double-strand-break-inducing agent activity at the variant recognition sites of f); and,
h) identifying a variant recognition site that results in an increased activity of the rare-cutting engineered double-strand-break-inducing agent when compared to the activity at the intended recognition site.
46) The method of embodiment 45 wherein the rare-cutting engineered double-strand-break-inducing agent is a Cas endonuclease
47) The method of embodiment 46 wherein the Cas endonuclease is capable of forming a complex with a guide RNA, wherein said complex enables the Cas endonuclease to introduce a double strand break into said genomic DNA.

### EXPERIMENTAL

### Example 1

### Creation of Rare Cutting Engineered Meganucleases

### A. LIG3-4 Meganuclease and LIG3-4 Intended Recognition Sequence

An endogenous maize genomic target site comprising the LIG3-4 intended recognition sequence (SEQ ID NO: 13) was selected for design of a rare-cutting double-strand break inducing agent (SEQ ID NO: 1) as described in US patent publication 2009-0133152 A1 (published May 21, 2009). The LIG3-4 intended recognition sequence is a 22 bp polynucleotide having the following sequence:

(SEQ ID NO: 13)
ATATACCTCACACGTACGCGTA.

### B. MHP14+ Meganucleases and MHP14 Recognition Site

An endogenous maize genomic target site comprising the MHP14+ intended recognition site (SEQ ID NO: 14) was selected for design of a rare-cutting double-strand break inducing agent (SEQ ID NO: 3) as described in U.S. patent application Ser. No. 13/427,138 filed on Mar. 22, 2012). The MHP14+ intended recognition site is a 22 bp polynucleotide located and having the following sequence:

(SEQ ID NO: 14)
CAAACAGATTCACGTCAGATTT.

### Example 2

Meganuclease Protein Production in E. coli

In order to produce purified protein for in vitro genomic and plasmid based meganuclease cleavage activity assays, DNA fragments corresponding to the open-reading-frames of Lig3-4 meganuclease (SEQ ID NO: 2) and MHP14+ meganuclease (SEQ ID NO: 4) were placed into a pQE80 (Qiagen) expression vector, transformed into BL21-Gold (Agilent Technologies) E. coli cells, and grown overnight on solid LB media containing 100 ppm of carbenicillin. Colonies were resuspended in 2 ml of 2XYT media and 250 μl of the cell suspension was used to inoculate a 50 ml culture of 2XYT supplemented with 100 ppm of carbenicillin. Cultures were grown at 37° C. for 1 to 1.5 hrs or until the OD600 reached 0.8 and then protein expression was induced by the addition of 0.5 ml of 100 mM IPTG. Cultures were cooled to room temperature and allowed to express protein for 2 hrs. Cells were pelleted by centrifuging for 10 minutes at 5,000 rcf. The supernant was decanted, the pellet resuspended in 1 ml of Buffer 1 (50 mM Tris-HCl (pH8.0), 500 mM NaCl, 10 mM imidizole), and transferred to a 1.5 ml microfuge tube. Cells were disrupted by sonication with a two-step ⅛″ microtip with 20 pulses (duty cycle 50, power 4) on a Branson 450 Analog Sonifer and centrifuged at 20,000 rcf for 15 minutes at 4° C. The supernant was diluted with 4 ml of Buffer 1 and loaded onto a disposable column containing 0.3 ml of Nickel-NTA Superflow resin (Qiagen). The column was washed with 5 ml of Buffer 2 (50 mM Tris-HCl (pH8.0), 500 mM NaCl, 60 mM imidizole) and the protein eluted with 0.6 ml of Buffer 4 (50 mM Tris-HCl (pH8.0), 500 mM NaCl, 250 mM imidizole) into a Vivaspin column (GE). To concentrate the samples, the vivaspin columns were centrifuged at 14,800 rcf for approximately 6 minutes or until the meniscus was between 75 and 50. A buffer exchange was performed using a Zeba Spin Desalting Column (Pierce) pre-equilibrated with storage buffer (25 mM Tris-HCl (pH8.0), 100 mM NaCl, 10 mM MgCl₂, 5 mM EDTA, 50% Glycerol). After the buffer exchange, Bovine Serum Albumin was added to a final concentration of 100 ng/μl and purified protein was stored at −20° C. until use.

### Example 3

### In Vitro Genomic DNA Cleavase Assays

To generate material for the capture of genomic variant recognition sites, in vitro assays were carried out with 114 nM of purified meganuclease protein isolated as described in Example 2 and 6.07 μg of purified maize genomic DNA at 32° C. for 80 minutes in a final volume of 80 μl in the presence of digestion buffer (50 mM Tris-HCl (pH 7.9), 100 mM NaCl, 10 mM MgCl₂, 1 mM DTT, 5 mM EDTA). After 80 minutes, the entire reaction was stopped with an equal volume of stop buffer (100 mM Tris-HCl (pH 8.0), 600 mM NaCl, 2% SDS, 100 mM EDTA, 1 mg of proteinase K per ml) and incubated at 50° C. for 30-45 minutes. Stopped reactions were purified by phenol/chloroform extraction and ethanol precipitated in the presence of 0.2 M NaCl. Precipitated genomic DNA was washed twice with 70% ethanol, dried, and resuspended in 34 μl of water.

Meganuclease protein concentration was determined visually on Nu-PAGE gels (Life Technologies) by calculating and then comparing band intensity with serially diluted samples of known concentration and genomic DNA concentration was determined using a Hoechst dye fluorometric assay.

To confirm cleavage (representing the % loss of meganuclease recognition sites) at the intended genomic recognition site, real-time PCR was performed on 1 μl of purified genomic DNA with a TaqMan assay spanning the meganuclease recognition site. The % cleavage or loss of meganuclease recognition sites was calculated via the ΔΔt method relative to an internal control TaqMan assay using the mock control as a calibrator.

### Example 4

### Capture of Genomic Variant Recognition Sites & Generation of Libraries for Illumina Deep Sequencing

Our method utilizes a novel adapter approach specifically tailored for the capture of cleaved I-CreI or engineered I-CreI homing endonuclease genomic variant recognition sites whose sequence is unknown and different in composition from the intended recognition site which is different from methods using restriction enzymes to perform reduced representation sequencing, restriction associated DNA (RAD-tag or RADseq) deep sequencing, whole genome sequencing (WGS), or genotype by sequencing (GBS).

Since the I-CreI homing endonuclease generates a 3′ 4 nucleotide overhang at the center of its 22 bp recognition site (+2, +1, −1, −2) upon cleavage (Thompson et al. (1992) Gene 119:247-51 and Durrenberger et al. (1993) Mol. Gen. Genet. 236:409-14) and has been demonstrated to cleave its recognition site in the context of different combinations of center 4 base pairs (+2, +1, −1, −2) (Molina et al. (2012) Nucleic Acids Res. 40:6936-45), adapters were generated containing a 3′ 4 nucleotide overhang containing all possible DNA nucleotide combinations (G, T, A, or C) of the overhang in an equimolar distribution. Thus, allowing for the efficient ligation and perfect complementation to all possible overhangs generated by recognition site cleavage in the genome subjected to I-CreI or engineered I-CreI homing endonuclease cleavage.

Similar strategies can be employed for other rare cutting double strand break inducing agents such as Zinc Finger and TALEN nucleases that cleave DNA with the non-specific catalytic domain of FokI generating overhangs of variable length and nucleotide composition within the intervening spacer region (Smith et al. (2000) Nucleic Acids Res. 28:3361-69 and Li et al. (2011) Nucleic Acids Res. 39:359-72). To capture genomic variant recognition sites, non-phosphorylated biotinylated adapters were synthesized and purified by HPLC (Integrated DNA Technologies, Inc.) containing a fully degenerate 4 nucleotide 3′ nucleotide overhang complementary to the 4 nucleotide 3′ overhang generated by meganuclease recognition site cleavage (SEQ ID NO: 5) and ligated to approximately 2 μg of meganuclease-cleaved genomic DNA (prepared as described in Example 3) in a 100 μl T4 ligase reaction (NEB) (representing a first adapter of the method to identify a variant recognition site for a rare-cutting engineered double-strand-break-inducing agent). Samples comprising the ligated DNA were then loaded in sonication microtubes and randomly sheared to an average peak size of 300 bps by sonication in a Covaris E220 system. The settings were 10% duty cycle, 140 peak incident power, and 200 cycles per burst. Fragments ranging from 200 to 500 bp were fractionated by electrophoresis in an agarose gel followed by gel extraction using the Qiagen Gel Extraction Kit according to manufacturer's recommendations.

Non-biotinylated ends were repaired using the End-It End repair kit (Epicentre) in a 75 μl reaction and column-purified (Qiagen). Single 3′ A overhang extension was performed by incubating the repaired DNA at 37° C. for 30 minutes in a 50 μl reaction containing ATP, 1×Klenow buffer (NEBnext), and 15 units Klenow (exo-). Samples were later purified by column (Qiagen) and ligated to indexed Illumina TruSeq-compatible adapters (representing a second set of adapters of the method to identify a variant recognition site for a rare-cutting engineered double-strand-break-inducing agent) in a 50 μl reaction containing 0.3 mM indexed adapter, 1× Quick ligation buffer and 5 units T4 DNA ligase (NEB) at room temperature. After ligation, samples were incubated at 65° C. for 15 minutes and the volume adjusted to 100 μl. Streptavidin magnetic capture was performed using Dynabeads M-280 streptavidin beads (Invitrogen). A total of 100 μl resuspended Streptavidin-Dynabeads (M-280) were washed twice in TE and resuspended in 100 μl 2×B&W buffer (10 mM Tris-HCL, 1 mM EDTA, 100 μl 0.5M EDTA, 2M NaCl). Samples were incubated at 30° C. for 30 minutes, the supernatant removed, and beads washed 4 times with 1 ml of 1×B&W buffer. The final enriched sample was resuspended in 30 μl EB buffer.

Fragments were recovered from the beads by 12-cycle PCR using Phusion master mix (NEB), in a 50 μl reaction in the presence of 0.4 pmol of recovery primer A (5′GTTGACATGCTGGATTGAGACTTC; SEQ ID NO: 6) and primer B (5′CAAGCAGAAGACGGCATACGA; SEQ ID NO: 7) according to manufacturer, except that an annealing temperature of 66° C. and extension time of 30 seconds were used. Recovered DNA was digested with SbfI (NEB) and purified twice with Agencourt AMPure XP Beads (SPRI) according to manufacturer instructions. The sample was ligated to an Illumina-compatible adapter with a SbfI compatible overhang (SEQ ID NO: 8). The supernatant was cleaned-up twice using Agencourt AMPure XP Beads, first using a 1:1.8 and then a 1:1 sample-to-bead ratio. The final samples were resuspended in 50 μl of EB buffer. A second amplification with the standard TruSeq PCR primer cocktail (Illumina) was performed, using a 60° C. annealing temperature, followed by clean-up twice using Agencourt AMPure XP Beads in a 1:1.8 and then a 1:1 sample-to-bead ratio. The final sample was resuspended in 20 μl. Samples were evaluated on a bioanalyzer, relatively quantified using qPCR with Illumina qPCR primers, and pooled. Prior to sequencing, the pools were size selected using the lab Xchip (Caliper), according to manufacturer's instructions.

### Example 5

### Non-Phosphorylated Adapters Enhance Enrichment of Meganuclease Cleaved Genomic Recognition Sites

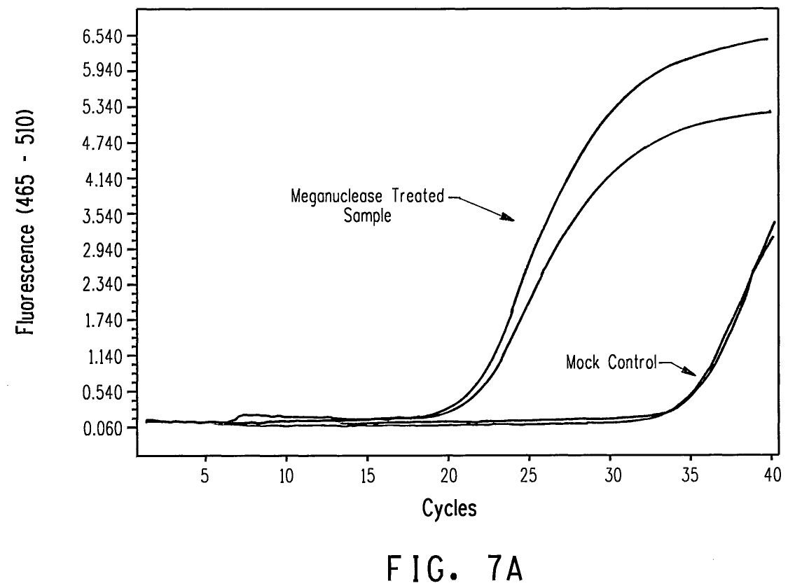

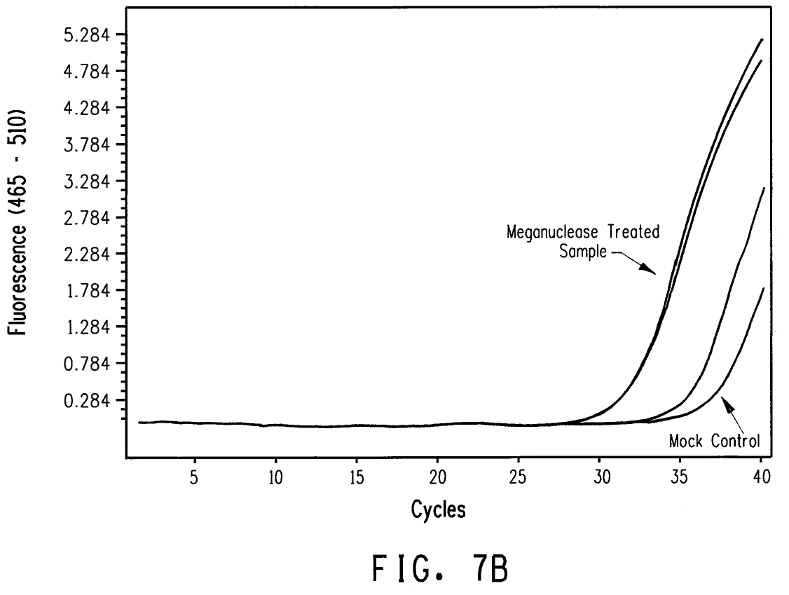

To examine the effect that phosphorylation has on the ability of a first adapter (as described in Example 4) to capture and enrich for meganuclease cleaved genomic recognition sites, libraries were made with both phosphorylated and non-phosphorylated adapters. After normalizing the DNA concentration of the libraries, they were examined for enrichment of the cleaved intended recognition site by real-time PCR with a TaqMan assay immediately adjacent to the intended recognition site. As shown in FIG. 7, both libraries demonstrated enrichment relative to the mock control but the amplification plots from the library generated with the non-phosphorylated adapter reached logarithmic amplification at a much earlier amplification cycle than the library generated with the phosphorylated adapter. Using standard curves derived from maize genomic DNA, the library generated with the non-phosphorylated adapter was estimated to be approximately 900 times more enriched for the left half of the intended recognition site than the library generated with the phosphorylated adapter.

### Example 6

### Illumina Deep Sequencing & Post-Run Trim

After the capture and enrichment for genomic variant recognition sites and DNA preparation as described in Example 4, cluster generation and paired-end read sequencing were performed on an Illumina cBot and Genome Analyzer IIx, respectively, according to the manufacturer's instructions. Approximately 30% (v/v) phiX DNA control (Illumina) was added to the DNA library solution prior to clustering. The random base composition of the phiX DNA fragments was expected to offset any base composition bias present at the vicinity of the meganuclease site. 100 cycle paired-end recipes were used on the Illumina Genome Analyzer. Sequences and quality scores were generated by the Illumina pipeline version 2.9 software for image analysis and base calling. During base calling, the phiX control data were used to obtain error estimates and re-calibrate raw quality scores for the other samples. After initial base calling, additional filtering was performed by the Illumina software where reads are excluded if the noise estimate exceeds thresholds defined by the Illumina base calling pipeline. Base call conversion to FASTQ format (using the Illumina CASSAVA software) was followed by additional filtering where reads were trimmed and filtered according to each base's respective quality score (where bases with a quality score below 10 are trimmed from the 3′ end of the read).

### Example 7

### In Silico Enrichment & Physical Mapping of Sequence Data to the Genomic Reference

To further enrich for meganuclease cleaved and adapter-ligated genomic DNA fragments, the set of reads from a sequencing experiment was filtered using a custom script that searches for read pairs or singletons wherein at least one member of the pair (or singleton) containing a sequence tag, GCAGGACGT (SEQ ID NO: 9), at the beginning of the read or its complement ACGTCCTGC (SEQ ID NO: 10) at the end of the read. Pairs or singleton reads matching this sequence are written to a new file for use in the mapping phase, while the rest are discarded.

To reunite both halves of the cleaved genomic recognition site, the enriched read set was physically mapped to the target reference genome using bowtie version 0.12.7 (Langmead B, Trapnell C, Pop M, Salzberg SL. Ultrafast and memory-efficient alignment of short DNA sequences to the human genome. Genome Biol 10:R25.) to identify the locations of the genome homologous to the reads. Alignment settings for specificity were adjusted on a case by case basis depending on the characteristics of the target genome and that genome's similarity to the source genotype of the data. The resulting alignments were then used for peak detection and identification of variant recognition sequences present in the genome.

### Example 8

### Peak Detection and Identification of Genomic Variant Recognition Site, and Composition

Using the genomic alignments generated in Example 7, peak detection was carried out using the MACs (Model-based analysis of ChiP-Seq) peak detection algorithm in Genedata Expressionist Refiner 7.5 (Genedata) with the parameters listed in Table 1.

TABLE 1
MACs (Model-based analysis of ChiP-Seq) peak detection
algorithm settings in Genedata Expressionist Refiner 7.5 (Genedata).

| | |
|---|---|
| Experiment Setup: | ChIP Samples with Control |
| Method: | MACS |
| Minimum Fold Enrichment | 10 |
| Maximum Fold Enrichment | 1.00E+09 |
| Bandwidth | 200 bp |
| p-Value Threshold | 1.00E−05 |

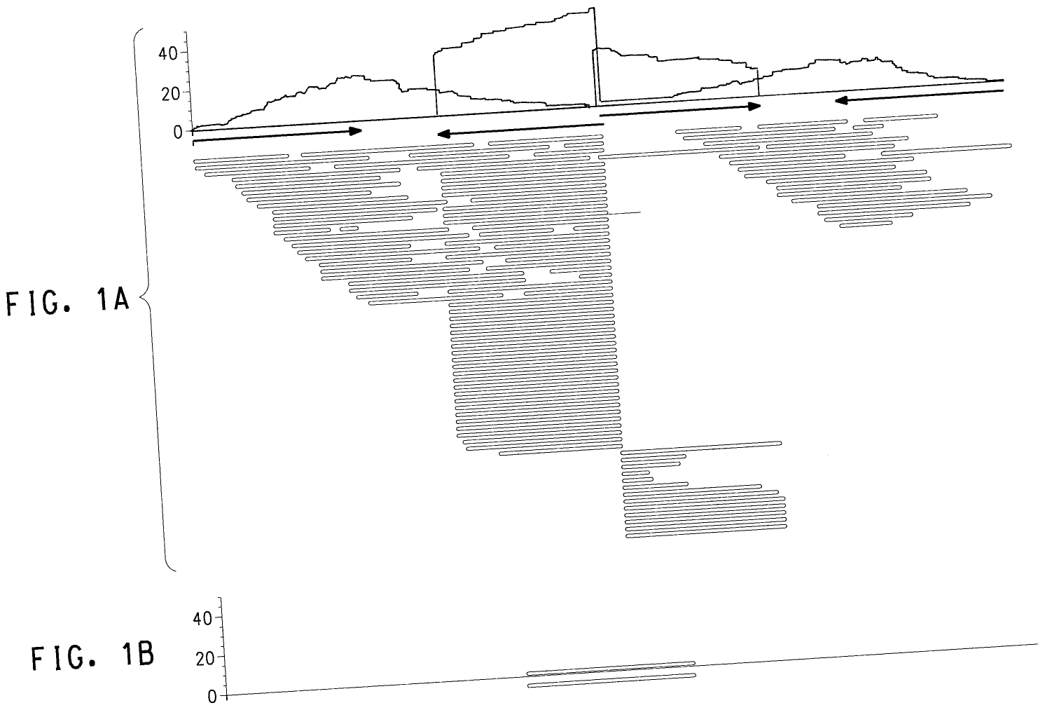

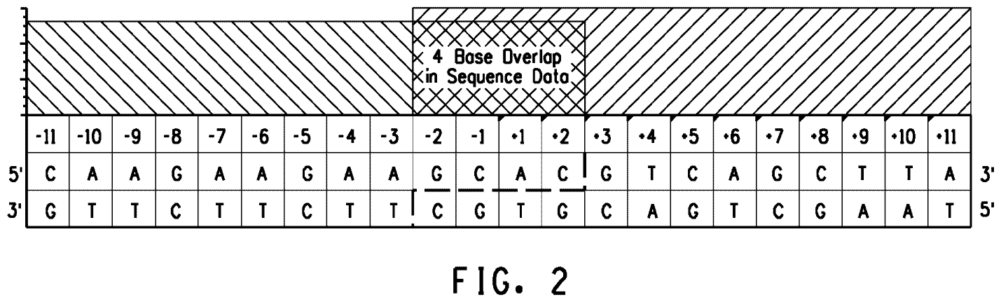

The chromosomal regions with significant enrichment relative to the mock control were exported to excel. Regions with the greatest difference between the treated and mock control samples were prioritized and confirmed as being enriched relative to the mock control and having a peak signature resulting from genomic DNA cleavage in the Genome Browser functionality of Genedata Expressionist Refiner 7.5. As shown in FIG. 1, recognition site peak signatures contain sequence data originating and diverging from the site of cleavage with the overlapping center corresponding to the overhang generated by the double-strand break inducing reagent. Based on the overhang defined by the peak signature, the precise variant recognition site sequence present in the genomic DNA could be identified as shown in FIG. 2. To determine the correct orientation of genomic variant recognition site sequences, they were aligned to the intended target site in both sense and anti-sense orientations with the best fitting orientation used as the recognition site sequence. A small proportion of genomic variant recognition sites fitted equally well in both orientations. These were left in the sense orientation. The oriented genomic variant recognition sites were aligned and the percent DNA nucleotide composition was calculated for each individual position of the recognition site and compared with the intended recognition site. As shown in FIG. 3, some positions in recognition sites exhibited an off-nucleotide preference; a preference for a nucleotide other than what was targeted in the intended recognition site.

### Example 9

### In Vitro Plasmid DNA Cleavase Assays

In vitro meganuclease cleavage activity can also be assayed using plasmid DNA, To compare meganuclease cleavage activity at intended and variant recognition sites, annealed oligonucleotides (synthesized by Integrated DNA Technologies, Inc.) containing the intended or variant recognition site with EcoRI and Hind/l overhangs were cloned into the Hind/l and EcoR/restriction endonucleases sites of the pBluescript SK+ plasmid (Stratagene, now an Agilent Technologies company), and in vitro DNA cleavage activity assayed as described in Example 3 with the following modifications. Timed digestions were carried out with 0.25 nM of linearized plasmid substrate containing a single intended or variant recognition site with 25 nM of purified meganuclease protein. In vitro assays were carried out at 37° C., 28° C., and 23° C. to best examine the cleavage activity at a given variant recognition site. Stopped reactions were purified with a Qiagen PCR purification column per the manufacturer's instruction and purified DNA was diluted 200-fold prior to the quantification of cleavage activity or % loss of recognition sites by qPCR.

### Example 10

### Identifying Variant Recognition Sites and Effect of Off-Nucleotide Preferences on Meganuclease Cleavase Activity

To assess the effect that the off-nucleotide preferences had on meganuclease cleavage activity, the off-nucleotide preferences were introduced into the intended recognition site individually and in combination (see FIG. 4), hereby generating variant recognition sites. Examples of such variant recognition sites are shown in Table 2 for the LIG3-4 meganuclease and Table 3 for the MHP14+ meganuclease.

TABLE 2
List of variant recognition sites for the LIG3-4 meganuclease. Nucleotides with an asterisk (*) indicate a modification compared to
the nucleotide at the corresponding location of the intended recognition site (SEQ ID NO: 13).

| | name of | | | | | | | | | | | | | | | | | | | | | | |
|---|---|---|---|---|---|---|---|---|---|---|---|---|---|---|---|---|---|---|---|---|---|---|---|
| SEQ | variable | | | | | | | | | | | | | | | | | | | | | | |
| ID | recognition | nucleotide position of reference nucleotide (SEQ ID NO: 13) | | | | | | | | | | | | | | | | | | | | | |
| NO: | site | 1 | 2 | 3 | 4 | 5 | 6 | 7 | 8 | 9 | 10 | 11 | 12 | 13 | 14 | 15 | 16 | 17 | 18 | 19 | 20 | 21 | 22 |
| 13 | intended | A | T | A | T | A | C | C | T | C | A | C | A | C | G | T | A | C | G | C | G | T | A |
| | LIG3-4 | | | | | | | | | | | | | | | | | | | | | | |
| 15 | −11C | C* | T | A | T | A | C | C | T | C | A | C | A | C | G | T | A | C | G | C | G | T | A |
| 16 | −7C | A | T | A | T | C* | C | C | T | C | A | C | A | C | G | T | A | C | G | C | G | T | A |
| 17 | −2G | A | T | A | T | A | C | C | T | C | G* | C | A | C | G | T | A | C | G | C | G | T | A |
| 18 | −1T | A | T | A | T | A | C | C | T | C | A | T* | A | C | G | T | A | C | G | C | G | T | A |
| 19 | +8T | A | T | A | T | A | C | C | T | C | A | C | A | C | G | T | A | C | G | T* | G | T | A |
| 20 | −7C, +8T | A | T | A | T | C* | C | C | T | C | A | C | A | C | G | T | A | C | G | T* | G | T | A |
| 21 | −11C, −7C, | C* | T | A | T | C* | C | C | | | | | | | | | | | | | | | |
| | −2G, −1T, | | | | | | | | | | | | | | | | | | | | | | |
| | +8T | | | | | | | | | | | | | | | | | | | | | | |

1.-11. (canceled)

12. A method for introducing into the genome of a cell a variant recognition site for a rare-cutting engineered double-strand-break-inducing agent capable of introducing a rare double strand break in an intended recognition site, said method comprising:
a. providing a donor DNA comprising a variant recognition site for a rare-cutting engineered double-strand-break-inducing agent capable of introducing a double-strand break in an intended recognition site, wherein said a rare-cutting engineered double-strand-break-inducing agent is also capable of introducing a double-strand break in said variant recognition site;
b. providing a plant cell;
c. contacting the plant cell with the donor DNA; and,
d. identifying at least one plant cell from (c) comprising in its genome said variant recognition site.

13. The method of claim 12 wherein the rare-cutting engineered double-strand-break-inducing agent is selected from the group consisting of a meganuclease, a zinc finger nuclease, a TAL effector nuclease, a transposase, a Cas endonuclease and a site-specific recombinase.

14.-42. (canceled)
